(19) United States
(12) Patent Application Publication
(10) Pub. No.: US 2023/0226293 A1
(43) Pub. Date: Jul. 20, 2023

(54) LUNG AIRWAY CLEARANCE

(71) Applicant: Tel HaShomer Medical Research Infrastructure and Services Ltd., Ramat-Gan (IL)

(72) Inventors: Moshe ASHKENAZI, Ganei-Tikva (IL); Gil SOKOL, Ganey-Tikva (IL); Ori EFRATI, Herzliya (IL)

(73) Assignee: Tel HaShomer Medical Research Infrastructure and Services Ltd., Ramat-Gan (IL)

(21) Appl. No.: 17/919,770

(22) PCT Filed: Apr. 22, 2021
(86) PCT No.: PCT/IL2021/050457

§ 371 Date: Oct. 19, 2022

Related U.S. Application Data
(60) Provisional application No. 63/013,614, filed on Apr. 22, 2020.

(51) Int. Cl. A61M 16/00

(52) U.S. Cl. CPC A61M 16/022; A61M 16/0003; A61M 2016/0027

(57) ABSTRACT

A system for providing treatment adapted to clear lung airways, the system including at least one pressure applicator adapted, when activated, to apply pressure at at least one specific location on a torso of a patient, and, when deactivated, to release the pressure, a sensor for sensing a signal associated with the patient, and, a controller, in communication with the sensor, adapted to analyze the signal and to control activation and deactivation of the at least one pressure applicator based, at least in part, on analyzing the signal. Related apparatus and methods are also described.

### RELATED APPLICATION/S

This application is a PCT application claiming priority from U.S. Provisional Patent Application No. 63/013,614, filed on 22 Apr. 2020.

The contents of the above application is incorporated by reference as if fully set forth herein.

### FIELD AND BACKGROUND OF THE INVENTION

The present disclosure, in some embodiments thereof, relates to a method and system for applying pressure on a patient's chest, in order to clear the patient's airways and/or assist in removing secretions within at least one lung.

Respiratory failure is common among patients with severe pneumonia or pneumonitis due to infection (viral, bacterial, parasitic, etc.). A first line of treatment is based on oral therapy and possibly oxygen, but an end stage of the treatment is sedating the patient, intubating the patient and ventilating the patient with a ventilation machine and by that, neutralizing an important defense mechanism of the lung—the cough. The ventilation machine supplies the patient with a certain volume or pressure of air, most of the time enriched with Oxygen, and enables gas exchange. The clinical features of the patient's lungs with severe infection in the lower airways are usually an inhomogeneous damage, areas with hyper-inflation, and areas with atelectasis (collapse of the lung), excessive secretions (sputum, pus) and narrowed airways (which means higher airway resistance). The inhomogeneous damage leads to an unequal air distribution. The ventilation machine supplies the patient with an air volume that is usually smaller or equal to the tidal breathing (typically 500 ml in adults). The amount of air supplied by the ventilating machine is typically enough during normal periods with healthy lungs, but during severe illness a vast majority of the air stays in the large airways and hardly reaches a periphery of the lung. So, in combination with the narrowed airways and the excessive sputum amount, the patient can't expectorate and the result is an excessive VP (ventilation perfusion) mismatch that leads to prolongation of the patient's duration with the ventilator.

Additional background art includes:
U.S. Patent Application Publication No. 2016/0113839;
U.S. Patent Application Publication No. 2010/0326442;
U.S. Pat. Nos. 10,555,870;
U.S. Pat. No. 10,518,048;
U.S. Pat. No. 9,687,415;
U.S. Pat. No. 8,578,939;
U.S. Pat. Nos. 7,909,034;
U.S. Pat. No. 7,594,508;
U.S. Pat. No. 6,461,315;
U.S. Pat. No. 6,415,791;
U.S. Pat. No. 4,928,674; and
U.S. Pat. No. 4,424,806.

The disclosures of all references mentioned above and throughout the present specification, as well as the disclosures of all references mentioned in those references, are hereby incorporated herein by reference.

### SUMMARY OF THE INVENTION

The present disclosure, in some embodiments thereof, relates to a method and system for applying pressure on a patient's chest, in order to clear the patient's airways and/or assist in removing secretions within at least one lung, and, more particularly, but not exclusively, to a vest for applying pressure on an outside of a patient's chest.

In some embodiments, applying pressure is performed by a set of inflatable sacs, or pillows, placed on a patient's chest and surrounded by a vest.

In some embodiments, the vest sacs are inflated in a specific order, optionally synchronized with a patient's breathing and/or coughing and/or with the patient's ventilation machine.

According to an aspect of some embodiments of the present disclosure there is provided a system for clearing lung airways, the system including a vest for surrounding a patient's thorax, in some embodiments even below the thorax, and a plurality of inflatable sacs for placing between the patient's torso or below and the vest.

According to some embodiments of the disclosure, the material including the vest is rigid.

According to some embodiments of the disclosure, further including pockets for placing the inflatable sacs.

According to some embodiments of the disclosure, further including a second, inner soft vest including pockets for placing the inflatable sacs.

According to some embodiments of the disclosure, the inflatable sacs includes hook-and-loop attachment to corresponding hook-and-loop components included in the vest.

According to some embodiments of the disclosure, the inflatable sacs are configured to deflate in less than 0.5 seconds.

According to some embodiments of the disclosure, further including individual inflation pipes connected to inputs of each one of the inflatable sacs.

According to some embodiments of the disclosure, further including an inflation pump for inflating the inflatable sacs.

According to some embodiments of the disclosure, further including a controller for controlling inflation of the inflatable sacs.

According to an aspect of some embodiments of the present disclosure there is provided a method for clearing lung airways, the method including, placing inflatable sacs in a rigid vest surrounding a patient's torso, inflating the inflatable sacs in a specific order, for a specific duration, and deflating the inflatable sacs.

According to some embodiments of the disclosure, the inflating and the deflating are synchronized with a patient's breathing, whether assisted or not.

According to some embodiments of the disclosure, the inflating and the deflating are synchronized with operation of a ventilator ventilating the patient.

According to some embodiments of the disclosure, a single inflating cycle occurs over more than one inspirium and expirium cycle of the ventilator.

According to some embodiments of the disclosure, a single deflating cycle occurs in less than one inspirium or expirium cycle of the ventilator.

According to some embodiments of the disclosure, further including producing lower pressure in the patient's lungs than in the ventilator by rapid deflation of the inflatable sacs.

According to some embodiments of the disclosure, further including producing lower pressure in the patient's lungs than in the ventilator by deflation of the inflatable sacs in less than 0.5 seconds.

According to some embodiments of the disclosure, further including producing lower pressure in the patient's lungs than in the ventilator by deflation of the inflatable sacs in less than 0.25 seconds.

According to some embodiments of the disclosure, further including producing lower pressure in the patient's lungs than outside the patient's body by rapid deflation of the inflatable sacs.

According to some embodiments of the disclosure, the inflating and the deflating are under control of a programmable controller.

According to some embodiments of the disclosure, the controller receives input of sensor measurement, and controls the inflating and deflating based on the measurement.

According to some embodiments of the disclosure, the sensor is a microphone.

According to some embodiments of the disclosure, the sensor is a microphone performing lung auscultation, and the controller is programmed to detect one or more states selected from a group consisting of air ventilation, wet crackles, dry crackles, fine crackles, secretion transport, wheezing, and cough.

According to some embodiments of the disclosure, the controller is programmed to select a treatment method based on the sensor measurement.

According to some embodiments of the disclosure, the sensor includes a plurality of sensors, arranged symmetrically relative to the vest. According to some embodiments of the disclosure, the plurality of sensors includes at least three sensors on each side of the patient's torso. According to some embodiments of the disclosure, the sensor is a volumeter, to measure volume of an adjacent space.

According to some embodiments of the disclosure, the inflating and the deflating are performed for each side of the patient's torso separately. According to some embodiments of the disclosure, the inflating and the deflating are performed for each inflatable sac separately. According to some embodiments of the disclosure, the inflatable sacs are deflated in less than 0.5 seconds.

According to some embodiments of the disclosure, locations for the placing inflatable sacs in the rigid vest surrounding a patient's torso are determined by image processing of an image of the patient's lungs.

According to some embodiments of the disclosure, a program for inflating the inflatable sacs in a specific order, for a specific duration, and deflating the inflatable sacs is determined by image processing of an image of the patient's lungs.

According to an aspect of some embodiments of the present disclosure there is provided a system for providing treatment adapted to clear lung airways, the system including a plurality of pressure applicators adapted, when activated, to apply pressure at a predetermined location of a torso of a patient, and, when deactivated, to release the pressure, a wearable component for locating a first one of the pressure applicators at a first location next to the torso and a second one of the pressure applicators at a second location next to the torso, and, a controller, adapted to control the activation and deactivation of the pressure applicators.

According to some embodiments of the disclosure, the first location is at a higher portion of the torso and the location is at a lower portion of the torso.

According to an aspect of some embodiments of the present disclosure there is provided a system for providing treatment adapted to clear lung airways, the system including at least one pressure applicator adapted, when activated, to apply pressure at at least one specific location on a torso of a patient, and, when deactivated, to release the pressure, a sensor for sensing a signal associated with the patient, and, a controller, in communication with the sensor, adapted to analyze the signal and to control activation and deactivation of the at least one pressure applicator based, at least in part, on analyzing the signal.

According to some embodiments of the disclosure, the activation and deactivation of the at least one pressure applicator includes controlling a duration or an amount of pressure, or a combination thereof, exerted by the at least one pressure applicator.

According to some embodiments of the disclosure, including a wearable component, wherein the pressure applicator is sized and shaped to be mounted on the wearable component. According to some embodiments of the disclosure, the controller is adapted to activate and deactivate at least one of the pressure applicators separately from another pressure applicator.

According to some embodiments of the disclosure, the sensor includes multiple sensors. According to some embodiments of the disclosure, the sensor includes a sensor selected from a group consisting of a pressure sensor, a microphone, a volumeter, an impedance sensor, an imaging system, a strain gauge, sensors for lung auscultation, and a combination thereof.

According to some embodiments of the disclosure, the controller is adapted to detect a physiological state of the patient based on analyzing the signal.

According to some embodiments of the disclosure, the physiological state is a physiological state selected from a group consisting of a stage within a breathing cycle of the patient, coughing of the patient, air ventilation, wet crackles, dry crackles, fine crackles, secretion transport, wheezing, intention to cough and a combination thereof.

According to some embodiments of the disclosure, the controller is adapted to provide guidance to a patient as to a required breathing pattern.

According to some embodiments of the disclosure, the controller is adapted to select guidance from a group consisting of long deep inspiration, shallow inspiration, prolonged expiration, prolong inhalation, quick breathing, and a combination thereof.

According to some embodiments of the disclosure, the guidance is based, at least in part, on the activation and deactivation of the at least one pressure applicator.

According to some embodiments of the disclosure, the guidance is based, at least in part, on the sensing.

According to some embodiments of the disclosure, the guidance is selected from a group consisting of written instructions, voiced instructions, sound instructions, sensory instructions, vibrations, visual indications, and a combination thereof.

According to some embodiments of the disclosure, mounting the at least one pressure applicator on the wearable component enables controlling a location where the pressure is exerted.

According to some embodiments of the disclosure, the at least one pressure applicator is selected from a group consisting of an inflatable sac, a fillable pad, an electrically activated pad, a manually adjustable belt, an automatically adjustable belt, a stretchable strap, and a combination thereof.

According to some embodiments of the disclosure, the controller receives data from a component selected from a group consisting of a BiPAP device, an invasive ventilator, non-invasive ventilator, a cough simulating device, and a combination thereof.

According to some embodiments of the disclosure, the controller synchronizes the activation and deactivation of the at least one pressure applicator with operation of the component.

According to some embodiments of the disclosure, release of pressure of the at least one pressure applicator is adapted to be provided in less than 0.5 seconds.

According to some embodiments of the disclosure, the system is configured to assist coughing by deactivation at least one of the at least one pressure applicator.

According to some embodiments of the disclosure, two of the pressure applicators are at least partially overlapping.

According to some embodiments of the disclosure, configured for positioning at least one pressure applicator on an abdomen of the patient.

According to some embodiments of the disclosure, including a database for storing data associated with the sensed signal.

According to some embodiments of the disclosure, the data is selected from a group consisting of a breathing pattern of the patient, a trend in the breathing pattern, an eigenvector of the breathing pattern, a shape of at least a portion of the breathing pattern, an area under at least a portion of the breathing pattern, a derivative of at least a portion of the breathing pattern, a number of coughs during a treatment, a cough pattern during a treatment, general compliance, a number of treatments the patient received in a period of time, and a combination thereof.

According to an aspect of some embodiments of the present disclosure there is provided a method for providing treatment adapted to clear lung airways, the method including, a. placing at least one pressure applicator on a patient's torso, b. sensing a signal associated with the patient, c. analyzing the signal, and d. performing a treatment protocol, the treatment protocol including activating and deactivating the at least one pressure applicator to apply and release pressure on the torso, based, at least in part, on the analyzing the signal, thereby providing treatment to clear lung airways.

According to some embodiments of the disclosure, including synchronizing the treatment protocol with a physiological state of the patient.

According to some embodiments of the disclosure, performing the treatment protocol includes activating and deactivating the at least one pressure applicator for specific durations of time or using specific amounts of pressure or a combination thereof.

According to some embodiments of the disclosure, performing the treatment protocol includes activating and deactivating the pressure applicators in a specific order.

According to some embodiments of the disclosure, the activating the at least one pressure applicator includes activating a fixed pressure for a duration longer than a period of time corresponding to one of a group selected from the patient's inspiration, the patient's expiration, and one breathing cycle of the patient.

According to some embodiments of the disclosure, the treatment protocol enables autogenic drainage.

According to some embodiments of the disclosure, including providing guidance to the patient to instruct the patient as to a desired breathing pattern.

According to some embodiments of the disclosure, the guidance is based on the activating and deactivating of the at least one pressure applicator.

According to some embodiments of the disclosure, the guidance is based on the sensing.

According to some embodiments of the disclosure, the guidance includes breathing instructions to the patient selected from a group consisting of long deep inspiration, shallow inspiration, prolonged expiration, prolong inhalation, quick breathing, and a combination thereof.

According to some embodiments of the disclosure, upon detection of a cough or intention to cough, at least one of the pressure applicators is deactivated.

According to some embodiments of the disclosure, including synchronizing the treatment protocol to the patient's breathing cycle.

According to some embodiments of the disclosure, the patient's intention to cough or coughing is detected automatically based on analyzing input from a sensor.

According to some embodiments of the disclosure, including automatically adjusting the treatment protocol based on the sensing.

According to some embodiments of the disclosure, the sensing includes data provided from a device selected from a group consisting of a BiPAP device, an invasive ventilator, a non-invasive ventilator, a cough stimulating device, and a combination thereof.

According to some embodiments of the disclosure, the treatment protocol is synchronized with the device based, at least in part, on the data.

According to some embodiments of the disclosure, including selecting the treatment protocol based, at least in part, on an image of the patient's lungs.

According to some embodiments of the disclosure, including placing the at least one pressure applicator on the patient's torso at a specific location based, at least in part, on an image of the patient's lungs.

According to some embodiments of the disclosure, including initially activating at least one of the pressure applicators for sensing the signal.

According to some embodiments of the disclosure, the treatment protocol includes activating at least one pressure applicator located next to a top of the patient's torso, applying pressure thereon, and activating at least one pressure applicator located next to a bottom of the patient's torso, applying pressure thereon.

According to some embodiments of the disclosure, further including deactivating the at least one pressure applicator located next to the bottom of the patient's torso, removing pressure therefrom.

According to some embodiments of the disclosure, including repeating the activating and deactivating the at least one pressure applicator located next to the bottom of the patient's torso a plurality of times.

According to some embodiments of the disclosure, including repeating activating at least one pressure applicator located next to a top of the patient's torso, applying pressure thereon, activating at least one pressure applicator located next to a bottom of the patient's torso, applying pressure thereon, deactivating the at least one pressure applicator located next to the bottom of the patient's torso, removing pressure therefrom, repeating the activating and deactivating the at least one pressure applicator located next to the bottom of the patient's torso a plurality of times, deactivating all of the pressure applicators, and repeating the above a plurality of times.

According to some embodiments of the disclosure, including increasing a transpulmonary pressure gradient in the patient's lungs by rapid deactivation of at least one pressure applicator.

According to some embodiments of the disclosure, the sensing includes performing lung auscultation.

According to some embodiments of the disclosure, including storing data from the sensing in a database.

According to some embodiments of the disclosure, including analyzing the data and performing at least one of providing guidance to the patient based, at least in part, on the analyzing, and adjusting the protocol based, at least in part, on the analyzing.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present disclosure may be embodied as a system, method or computer program product. Accordingly, some embodiments of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present disclosure may take the form of a computer program product embodied in one or more computer readable medium(s) having computer readable program code embodied thereon. Implementation of the method and/or system of some embodiments of the disclosure can involve performing and/or completing selected tasks manually, automatically, or a combination thereof. Moreover, according to actual instrumentation and equipment of some embodiments of the method and/or system of the disclosure, several selected tasks could be implemented by hardware, by software or by firmware and/or by a combination thereof, e.g., using an operating system.

For example, hardware for performing selected tasks according to some embodiments of the disclosure could be implemented as a chip or a circuit. As software, selected tasks according to some embodiments of the disclosure could be implemented as a plurality of software instructions being executed by a computer using any suitable operating system. In an exemplary embodiment of the disclosure, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as a physician or a medical technician, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

In the drawings:

FIG. 1A is a simplified illustration of a prior art system;

FIG. 1B is a simplified illustration of a system constructed and operational according to an example embodiment;

FIG. 1C is a simplified illustration of a system constructed and operational according to an example embodiment;

FIGS. 2A-2C are simplified illustrations of one or more inflatable sacs in relation to a patient, in a system constructed and operational according to an example embodiment;

FIG. 2D is a simplified graph illustration of Autogenic Drainage (AD) airway clearance therapy according to an example embodiment;

FIG. 2E is a simplified graph illustration of application of pressure by chambers of a vest synchronized with a breathing cycle, according to an example embodiment;

FIG. 2F is a simplified flow chart illustration of a treatment cycle according to an example embodiment;

FIG. 2G is a simplified flow chart illustration of a method of treatment according to an example embodiment;

FIG. 2H is a simplified flow chart illustration of a method of treatment according to an example embodiment;

FIG. 2I is a simplified flow chart illustration of a method of treatment according to an example embodiment;

FIG. 2J is a simplified flow chart illustration of a method of treatment according to an example embodiment;

FIG. 2K is a simplified flow chart illustration of a method of treatment according to an example embodiment;

FIGS. 3A-3D are simplified graphical illustrations of respiration and pressure at three pairs of inflatable sacs according to an example embodiment;

FIGS. 4A-4D are simplified graphical illustrations of respiration and pressure at three pairs of inflatable sacs according to an example embodiment;

FIG. 5 is a simplified graphical illustration of respiration and pressure at three inflatable sacs according to an example embodiment;

FIG. 6 is a simplified schematic illustration of a system constructed according to an example embodiment;

FIG. 7 is a simplified flow chart illustration of a method for clearing lung airways according to an example embodiment;

FIGS. 8A-8C are simplified illustrations of one or more inflatable sacs in relation to a patient, in a system constructed and operational according to an example embodiment;

FIGS. 9A-9C are simplified illustrations of one or more inflatable sacs in relation to a patient, in a system constructed and operational according to an example embodiment;

FIG. 10 is a simplified flow chart illustration of a method for providing treatment adapted to clear lung airways according to an example embodiment; and

FIG. 11 is a simplified block diagram illustration of a system for providing treatment adapted to clear lung airways according to an example embodiment.

### DESCRIPTION OF SPECIFIC EMBODIMENTS OF THE INVENTION

The present disclosure, in some embodiments thereof, relates to a method and system for applying pressure on a patient's chest, in order to clear the patient's airways, and, more particularly, but not exclusively, to a vest for applying pressure on an outside of a patient's chest.

### INTRODUCTION

Some diseases, such as for example acute or chronic lung diseases and even chronic lung diseases caused by the 2020 world wide COVID-19 pandemic, cause severe lung damage, potentially non-uniformly through the lung, and interferes with input and output of air from the lung. Phlegm builds up in the lung. In order to remove the phlegm one desires to cause coughing.

According to an aspect of some embodiments of the present disclosure there is provided a system for providing treatment adapted to clear lung airways, the system including:
a. at least one pressure applicator adapted, when activated, to apply pressure to at least one predetermined location of a torso of a patient; and, when deactivated, to remove said pressure from said at least one predetermined location;
b. at least one sensor for sensing at least one parameter associated with at least one physiological state of said patient;
c. a controller, in communication with said at least one sensor, adapted to control activation and deactivation of said at least one pressure applicator, based on the information received from said at least one sensor according to a predefined treatment protocol so as to synchronize the activation and deactivation of said at least one pressure applicator with said at least one parameter associated with at least one physiological state of said patient.

It should be noted that the physiological state of said patient is selected from a group including at least one stage within a breathing cycle of said patient, lung auscultation, coughing of said patient and a combination thereof.

According to an aspect of some embodiments of the present disclosure there is provided a method for providing treatment adapted to clear lung airways, the method including: (a) placing at least one pressure applicator on a predetermined location on a patient's torso; (b) activating and deactivation said at least one pressure applicator in a specific order, for a specific duration, to apply and release pressure on said torso respectively; thereby clearing lung airways.

According to an aspect of some embodiments of the present disclosure there is provided a system for providing treatment adapted to clear lung airways, the system including: (a) at least one inflatable sac adapted, when inflated, to apply pressure to at least one predetermined location of a torso of a patient; and, when deflated, to remove said pressure from said at least one predetermined location; (b) at least one sensor for sensing at least one parameter associated with at least one physiological state of said patient; and, a controller, in communication with said at least one sensor, adapted to control the inflation and deflation of said at least one inflatable sac, based on the information received from said at least one sensor according to a predefined treatment protocol so as to synchronize the inflation and deflation of said at least one pressure applicator with said at least one parameter associated with at least one physiological state of said patient.

It should be noted that the physiological state of said patient is selected from a group consisting of at least one stage within a breathing cycle of said patient, lung auscultation, coughing of said patient and any combination thereof.

According to an aspect of some embodiments of the present disclosure there is provided a method for providing treatment adapted to clear lung airways, the method comprising: (a) placing at least one inflatable sac on predetermined location on a patient's torso; and, inflating and deflating said at least one inflatable sac in a specific order, for a specific duration, to apply and release pressure on said torso respectively; thereby applying pressure on said predetermined location and clearing lung airways.

According to an aspect of some embodiments of the present disclosure there is provided a medical device, to enable airway clearance in patients with acute or chronic lung diseases.

In some embodiments, the device is a vest positioned on a patient's upper body and integrated with one or more actuator(s) for applying pressure on predefined locations thereon for a predefined period of time. For example, the vest can be shaped as a simple belt, such that tightening thereof results in application of pressure on said predefined locations on the patient's upper body. According to such embodiments, a strain gauge sensor is optionally utilized to quantify an amount of pressure in the lungs and an amount of pressure to be applied on the torso (or releases therefrom).

In some embodiments, integration of a plurality of electrically activated elements (e.g., pads, pistons etc.) is provided within a wearable vest, such that one or more or all elements can be activated independently to apply pressure.

In some embodiments, as described herein, can be a manually and/or automatically stretchable belt(s) or strap(s), such that, when positioned on the patient torso and stretched, apply pressure on the patient's torso.

In some embodiments, as described herein, integration of a plurality of fillable elements within a wearable vest is provided, such that one or more or all elements can be activated, for example filled with liquid, to apply pressure.

In some embodiments, the device is a vest positioned on a patient's upper body and includes an envelope and several inflatable sacs in an inner side of the vest that are controlled, optionally automatically, by a controller. The inflatable sacs are inflated and deflate at will, to potentially exert pressure on various areas of the patient's upper body, to assist in removing phlegm.

Other examples of application of pressure on the various areas of the patient's upper body may be by:
Inflating sacs with fluid (e.g. water);
Shortening straps surrounding the patient's upper body; and
Mechanically manipulating pads against the patient's upper body.

In some embodiments, a physiological principle upon which some methods are based is on breathing at different lung volumes to move secretions along the bronchial tree, such a technique is called Autogenic Drainage.

In some embodiments, by application of constant/dynamic pressure at predefined locations of the patient's upper body according to a predefined protocol (of e.g., time and amount of pressure applied), secretions are removed from the lungs; potentially clearing the airways.

According to some embodiments, sensors for sensing at least one parameter associated with one or more of a breathing cycle of the patient, lung auscultation and coughing of said patient are provided.

According to an aspect of some embodiments of the present disclosure such application of pressure is combined with provision of breathing instructions (hereinafter referred to as ‘guidance’ or ‘instruction’ to the patient). In some embodiments, such guidance is optionally provided to support autogenic drainage airway clearance technique. In some embodiments, the guidance is optionally synchronized with the patient's breathing data received from sensors.

According to some embodiments, the vest is integrated with at least one control adapted to control the operation of the pressure applicators (and/or the vest). In other words, the control may be adapted to control the amount of pressure applied, the timing thereof and the location (on the patient's body) at which the pressure is applied.

In some embodiments, the controller synchronizes with a ventilation machine, in case of a ventilated patient.

In some embodiments, the controller synchronizes with a patient's breathing cycle. In some embodiments, the vest is independent of a patient's interaction, e.g., it does not require a patient's cooperation. In some embodiments, the controller uses an algorithm based on physiological principles, and on experience of a respiratory physiotherapist.

In some embodiments, optionally based on data provided by the sensors, the system optionally provides guidance, optionally based on the monitoring, to the patient as to how to breathe, thereby enabling the patient to cooperate with and/or assist the treatment being provided. The guidance may be in form of one or more of displaying instructions (e.g., written and/or graphical illustration); providing audio instructions (e.g., voice commands or any acceptable sound to provide instructions); providing sensible instructions (e.g., vibration of at least one element selected from a group consisting of the wearable vest, any device in communication with said vest); and displaying visual indications for a desired type of breathing guidance to the patient as to how to breath.

It should be noted that according to some embodiments, a variety of biofeedback mechanisms can be employed. The biofeedback can be utilized to sense (by means of any sensor, in communication with the patient's body) and/or to provide feedback (guidance) to the patient based on the sensed information; or both.

The term “sac” and the term “pillow” and the term “pad” in all their grammatical forms are used throughout the present specification and claims interchangeably to mean a bladder which may be inflated or pressurized by filling with fluid or gas and deflated or depressurized by emptying the fluid or gas.

In some embodiments, the ‘sac’ or ‘pillow’ or ‘pad’ may refer to electrically activated elements (by means of, e.g., electrical motor) to apply mechanical pressure on the patient's upper body. According to another embodiment, said elements are hydraulic activated elements.

The term “deflate” in all its grammatical forms is used throughout the present specification and claims to mean opening a valve which enables release of pressure and/or actually pumping gas or fluid to deflate an inflatable sac and/or releasing the pressure applied by electrically activated elements or hydraulically activated elements.

One potential solution to improving ventilation in airways can be increasing the ventilation pressure. However, in such a case one risks the patient with barotrauma such as pneumothorax, which may potentially lead to a significant increase in ventilation duration.

In some embodiments, an effective airway clearance potentially serves as a complementary treatment and potentially enables to lower VP (Ventilation Perfusion) mismatch and shorten the ventilation duration of a patient. Ventilation and perfusion are optionally adjusted to each other in order to enable adequate oxygenation of the blood.

In some embodiments, a vest and/or a pressure application method (e.g., inflation and deflation elements) is intended to enable airway clearance in ventilated patients where the patients can potentially use feedback and/or instruction from the system, and change breathing accordingly.

In some embodiments, a vest and/or a pressure application method (e.g., inflation and deflation elements) is intended to enable airway clearance in ventilated patients with no cooperation from the patients.

In some embodiments, a vest and/or a pressure application method (e.g., inflation and deflation elements) is intended to enable airway clearance in patients with acute or chronic lung diseases, such as chronic obstructive lung diseases (COPD), Cystic Fibrosis (CF), Bronchiectasis, optionally including ventilated patients with corona virus pneumonitis. In some embodiments, the device does not require a patient's cooperation, so, the device is also suitable for sedated, ventilated patients. In some embodiments, the vest is optionally positioned on a patient's torso. In some embodiments, the vest is optionally synchronized with a ventilation machine. In some embodiments, a purpose of the vest is to potentially enable airway clearance in a patient with one or more of lung debilitating conditions, such as pneumonia, pneumonitis and ARDS (Acute Respiratory Disease Syndrome).

In some embodiments, the device potentially enables expectoration of a sedated patient by ventilating distal and closed areas of the lungs, potentially without increasing ventilation pressure.

According to some embodiments the orientation of the patient is selected from a group including a sitting position, a standing position, a lying position (e.g., lying on the back, on the stomach and lying on the side) and any combination thereof.

The term “ventilator” in all its grammatical forms is used throughout the present specification and claims interchangeably with the terms “PAP (Positive Airway Pressure) device”, “BiPAP device” and “cough simulating device” and its corresponding grammatical forms.

The term “ventilator” in all its grammatical forms is used throughout the present specification and claims to mean:

Invasive ventilators and non-invasive ventilators (NIV). Examples for of NIV include PAP devices, BiPAP devices, mechanical insufflation-exsufflation devices, and cough stimulators; and

positive pressure devices and negative pressure devices such as a suction device.

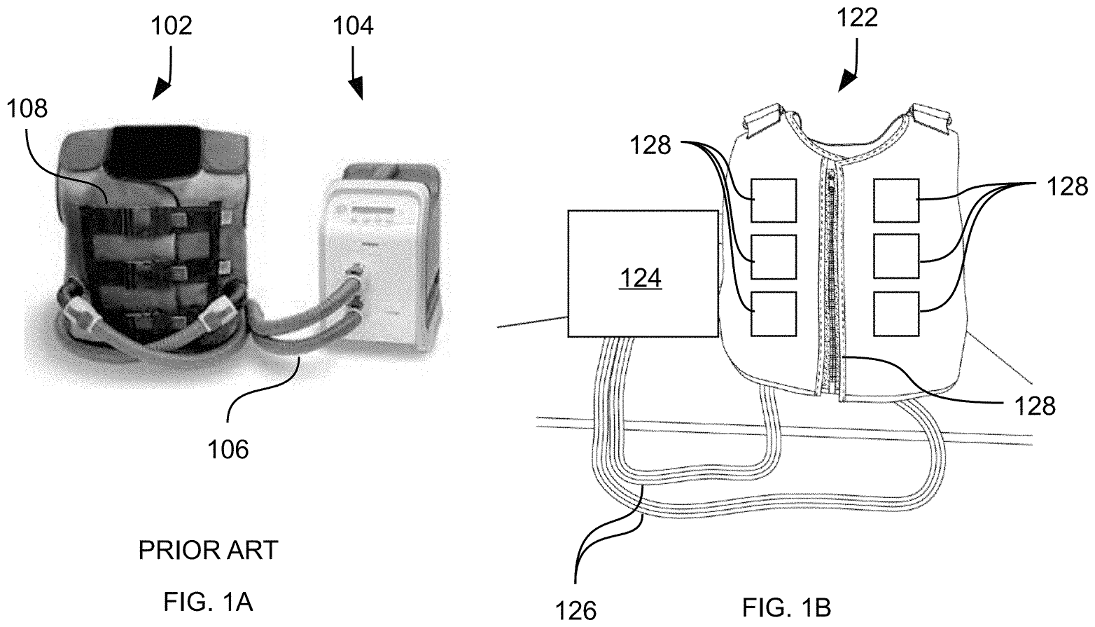

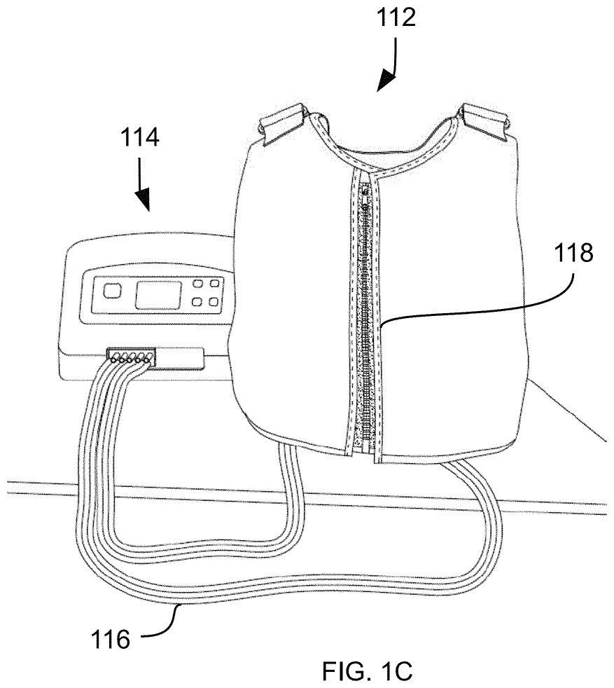

For purposes of better understanding some embodiments of the present disclosure, as illustrated in FIG. 1B and onward of the drawings, reference is first made to the construction and operation of a simplified illustration of a prior art system as illustrated in FIG. 1A. FIG. 1A shows a non-limiting example of a vest and vest inflation system in order to set a context for various descriptions provided herein.

FIG. 1A shows a vest 102 opening at a front, and straps 108 for closing the vest 102.

FIG. 1A also show an inflator 104 or inflating pump 104 and pipes 106 which lead to the vest, apparently one pipe to each side of the vest.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

Before explaining at least one embodiment of the disclosure in detail, it is to be understood that the disclosure is not necessarily limited in its application to the details set forth in the following description or exemplified by the Examples. The disclosure is capable of other embodiments or of being practiced or carried out in various ways.

Producing pressure on a patient's torso can potentially cause air from a healthier area of a lung to move to a more-diseased area, potentially enabling clearing secretions from airways, for example by causing opening and/or air movement in small airways, movement of the secretions, and/or causing coughing or suction to evacuate the secretions; to potentially clear the airways.

Reference is now made to FIG. 1B, which is a simplified illustration of a system constructed and operational according to an example embodiment.

FIG. 1B shows a non-limiting example of a vest 122 and vest pressure applicators 128.

In various embodiments, the pressure applicators 128 may be electrically driven mechanical pads; pneumatically driven pads or inflatable sacs; and hydraulically driven pads or inflatable sacs.

FIG. 1B shows the vest 122, in the non-limiting example of FIG. 1B opening at a front, and a zipper 118 for closing the vest 122. It is noted that in other embodiments the vest may be opened at a back or opened at one or both sides.

FIG. 1B also show a powering unit 124 and powering pipes or wires 116.

In some embodiments, the powering unit 124 may be an electrical power supply and/or controller 124, and FIG. 1B also shows optional electrical wires 126 which lead to the vest 122.

In some embodiments, the powering unit 124 may be a source for air pressure and/or controller 124 and FIG. 1B also shows pipes or tubes 126 which lead to the vest 122.

In some embodiments, the powering unit 124 may be a source for hydraulic pressure and/or pump and/or controller 124 and FIG. 1B also shows optional pipes or tubes 126 which lead to the vest 122.

FIG. 1B shows multiple wires/tubes 126. The number of wires/tubes is not limited to 6 as shown in FIG. 1B.

In some embodiments, one or several pressure applicators 128 may be within the vest. In some embodiments, the number of wires/tubes 126 may be the same as the number of pressure applicators 128 in the vest.

In some embodiments, one wire/tube may provide power to one associated pressure applicator 128. In some embodiments, one wire/tube may provide power to more than one pressure applicator 128.

FIG. 1B shows two wire/tube groups, one group to each side of the vest 122. However, it should be noted that the same group of wires/tubes can be for both sides of the vest.

In some embodiments, the powering unit 124 may be a liquid storage unit 124 and the tubes/pipes 126 lead liquid (for example water or oil) to the vest 122.

Reference is now made to FIG. 1C, which is a simplified illustration of a system constructed and operational according to an example embodiment.

FIG. 1C shows a non-limiting example of an air-driven vest and a vest inflation system.

FIG. 1C shows a vest 112, in the non-limiting example of FIG. 1C opening at a front, and a zipper 118 for closing the vest 112. It is noted that in other embodiments the vest may be open at a back or opening at one or both sides.

FIG. 1C also show an inflator 114 or inflating pump 114 and pipes 116 which lead to the vest 112.

FIG. 1C shows multiple pipes 116. The number of pipes is not limited to the 6 pipes shown in FIG. 1C. In some embodiments, one or several inflatable sacs may be within the vest. In some embodiments, the number of pipes 116 may be the same as the number of inflatable sacs in the vest. In some embodiments, one pipe may provide air to one associated inflatable sac. In some embodiments, one pipe may provide air to more than one inflatable sac.

FIG. 1C shows two pipe groups, one pipe group to each side of the vest 112.

Directing air to inflate the sacs, or releasing air, may be performed by valves under control of a controller, and/or by a pipe to one or more sacs and valves associated with the pipes, optionally also under control of a controller.

A high-level device description of some example embodiments

An aspect of some embodiments relates to a personalized chest physiotherapy system designed to provide efficient airway clearance for patients with chronic lung diseases, in a clinical or hospital setting, or independently at their home.

In some embodiments, the device is a vest positioned on a patient's upper body, and means for applying pressure on predefined locations thereon for a predefined period of time. For example, the vest can be shaped as a simple belt, such that tightening thereof results in application of pressure on said predefined locations on the patient's upper body.

In some embodiments, as described herein, includes integration of a plurality of electrically activated elements within a wearable vest, such that each element is activated independently to apply pressure.

In some embodiments, application of pressure on the various areas of the patient's upper body may be exerted by:
Inflating sacs with fluid (e.g. water);
Shortening straps surrounding the patient's upper body; and
Mechanically manipulating pads against the patient's upper body.

According to some embodiments, the system includes a vest with independently inflatable air chambers, which delivers a sequence of chest compressions, to promote breathing at different lung volumes and locations.

In some embodiments, sensors integrated with the system synchronize the activation with a patient's breathing cycle, and/or with use of invasive or non-invasive ventilation. By way of a non-limiting example such a non-invasive ventilation may be a BiPAP device.

In some embodiments, the synchronization is optionally done by pressure sensors that track pressure in one or more designated air chamber(s). Chest movement while breathing potentially results in pressure changes in the chamber, and the user's breathing pattern is optionally detected based on a sampled pressure signal.

In some embodiments, breathing cycle detection potentially enables to synchronize the application of pressure (e.g., inflation and deflation of vest chambers) with inhalation and exhalation.

In some embodiments, breathing cycle detection potentially enables detecting when a patient coughs.

In some embodiments, breathing cycle detection potentially enables detecting when a patient removes a breathing mask, for example a BiPAP mask.

In some embodiments, the vest is optionally controlled to deflate the vest and pause the treatment when a patient is detected to remove a breathing mask or detected to begin a cough cycle.

In some embodiments, a patient is optionally assisted in coughing by timing of inflation and/or deflation of air sacs in the vest.

In some embodiments, the system optionally provides feedback and/or guidance during treatment to instruct a patient as to a desired breathing pattern according to a treatment algorithm—by way of a non-limiting example guide the patient when to take long deep inspiration as oppose to shallow inspiration, when to prolong expiration, when to prolong inhalation, when to perform quick breathing.

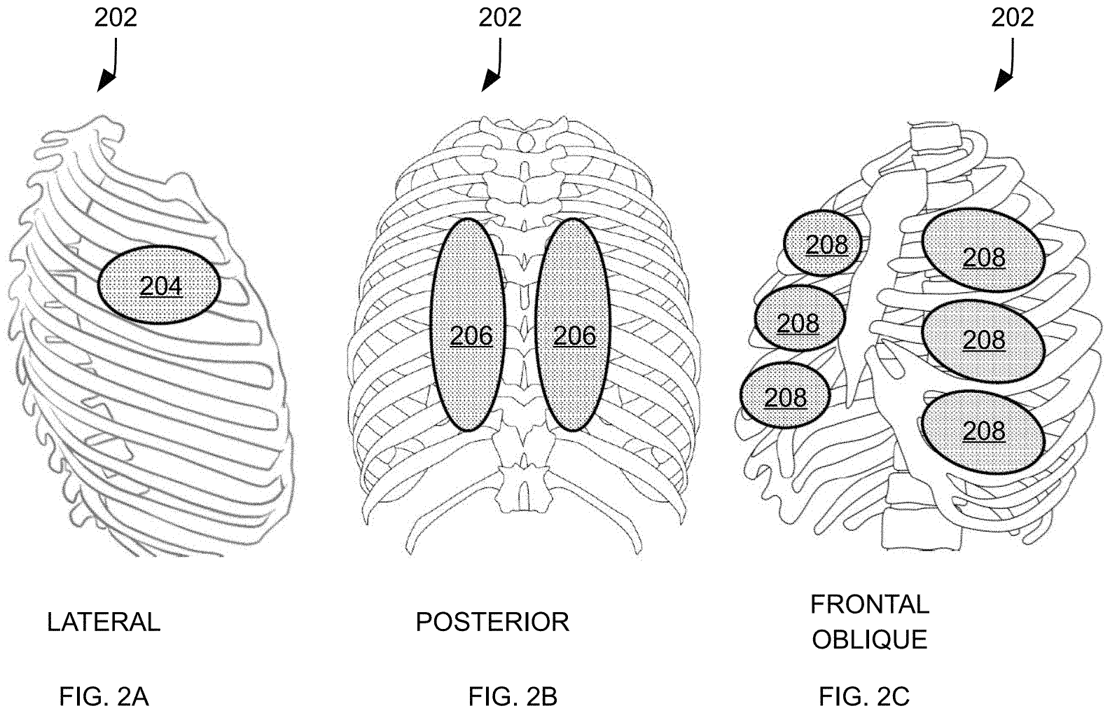

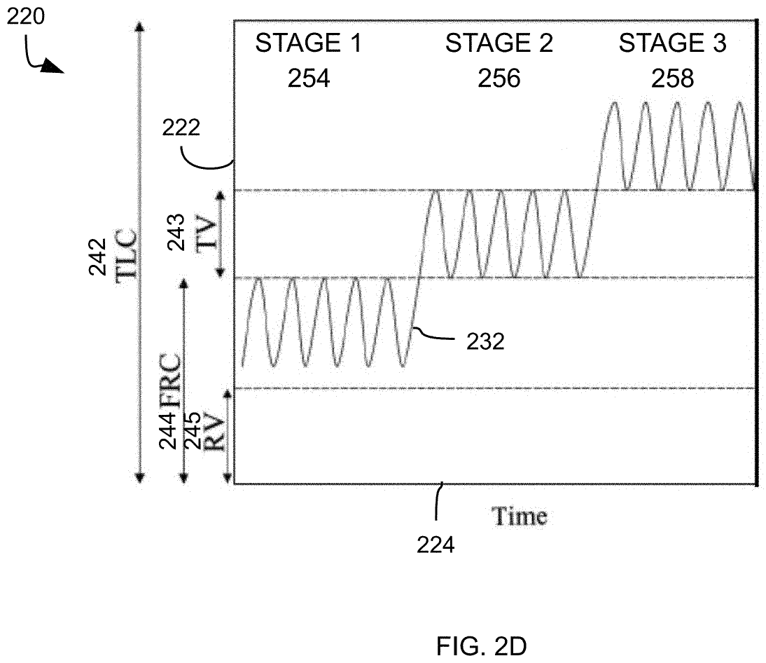

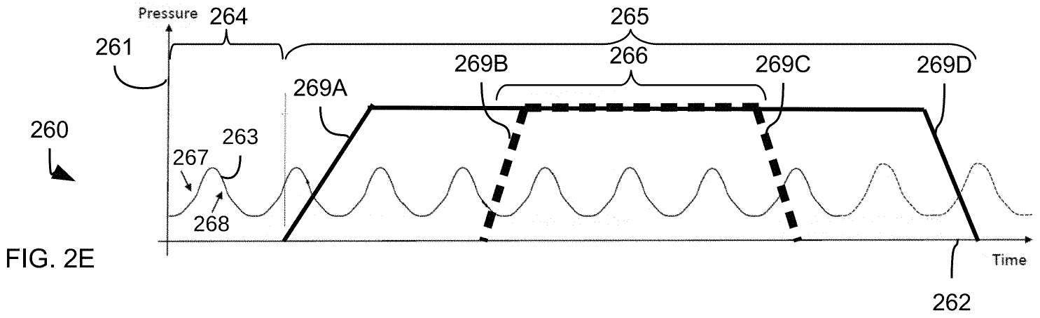

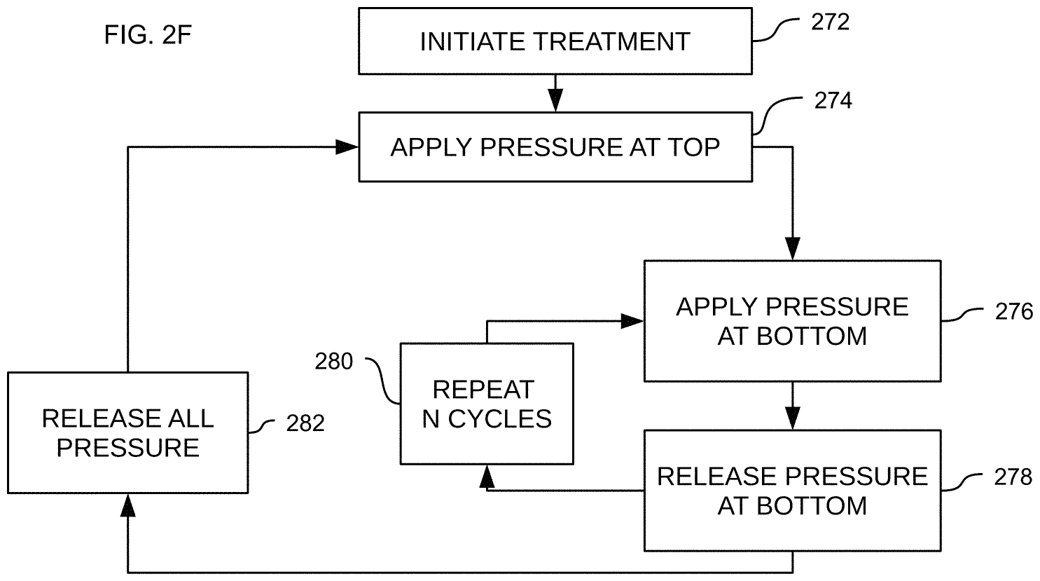

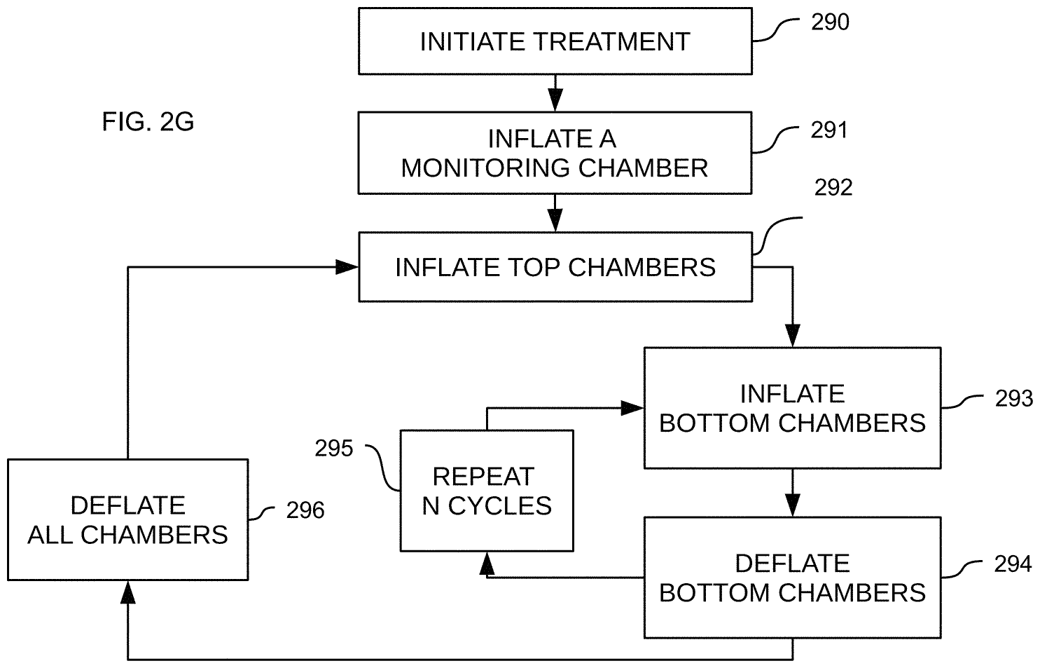

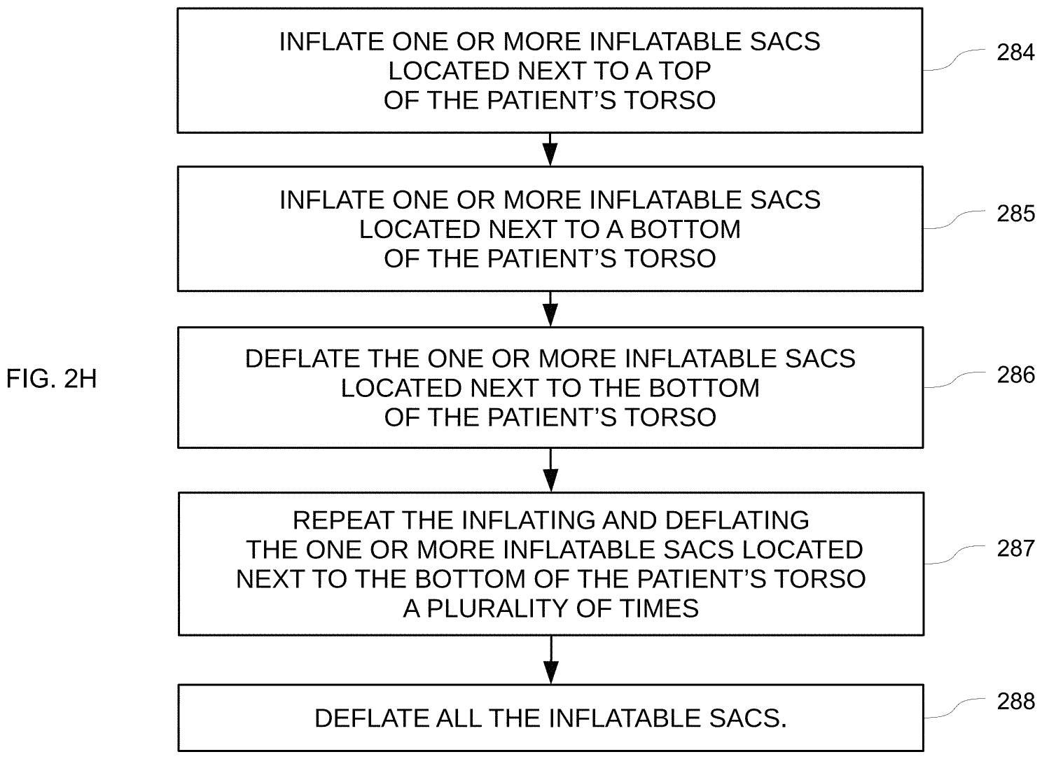

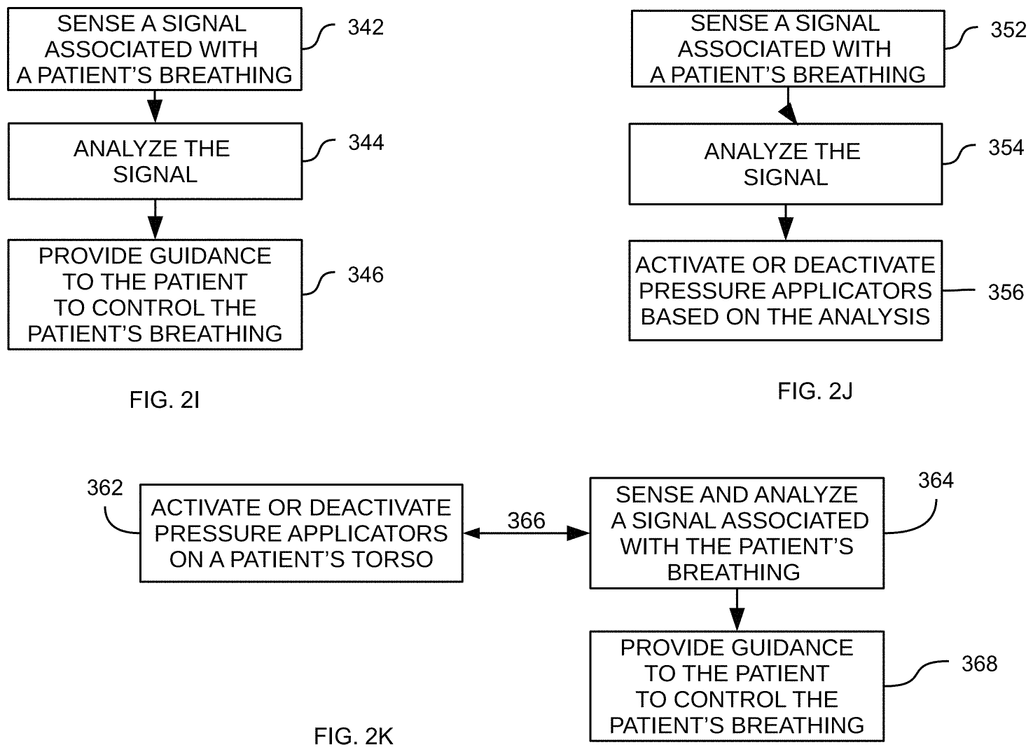

Reference is now made to FIGS. 2A-2C, which are simplified illustrations of one or more inflatable sacs in relation to a patient, in a system constructed and operational according to an example embodiment.

FIGS. 2A-2C show non-limiting examples of a patient and one or more inflatable sac(s) in order to set a context for various descriptions provided herein.

FIGS. 2A-2C do not show the vest (such as shown in FIGS. 1B and 1C), so that the vest does not obscure a view of the sacs.

FIG. 2A shows a lateral view of a skeleton 202 representing a patient, and an inflatable sac 204 placed on a first example location, on a side of the patient.

FIG. 2B shows a posterior view of the skeleton 202 representing a patient, and two inflatable sacs 206 placed on two example locations, on a back of the patient, one of each side.

FIG. 2C shows a frontal oblique view of the skeleton 202 representing a patient, and six inflatable sacs 208 placed on six example locations, on a front of the patient.

It is noted that sacs may be placed at locations selected by a physician and/or a physiotherapist, and/or a medical technician, optionally according to locations selected to apply pressure on sections of a lung from which secretions are to be cleared, and/or in which airways are to be cleared.

In some embodiments, the sections of the lung are optionally selected based on imaging of a patient's lungs.

In some embodiments, sacs are optionally placed at fixed locations, and several models or instances of a vest with sacs are provided for a physician to select for a patient, based on patient size, gender, and additional physical considerations.

It is noted that the sac locations shown in FIGS. 2A-2C are not intended to be limiting, but rather intended as non-limiting examples.

An aspect of some embodiments includes using a vest for applying pressure on an outside of a patient's chest.

The vest includes at least one element adapted to apply pressure on the patient's body.

As described above, the pressure application can be e.g., inflatable sacs (such that when inflated with air, the same apply pressure), fluid-fillable elements (such that when filled with liquid, the same apply pressure), mechanically activated elements, a simple belt being applied to the patient torso and stretched (to apply pressure thereon) etc.

In some embodiments, the vest is optionally used in parallel with ventilation of a patient's lungs.

In some embodiments, the vest is optionally used in parallel with collateral ventilation of the lungs.

Various descriptions herein refer to inflatable/deflatable sacs. However, other applications of pressure can be utilized, as described herein.

According to some embodiments, the system include a control unit with an air compressor and an inflatable vest that connects to the air compressor. The inflatable vest includes one or more air chamber(s), connected to the air compressor by pneumatic tubes.

In some embodiments, the air chambers are optionally inflated sequentially, to apply localized external pressure on a patient's chest in order to promote breathing at different lung volumes.

In some embodiments, adjusting depth and/or location of lung volumes during respiration potentially generates shearing forces induced by airflow, which potentially loosen and/or mobilize and/or move secretions from peripheral towards central airways. Once the secretions or mucus has moved to the larger airways, it can potentially be expelled by coughing.

In some embodiments, the vest includes a zipper on the front. In some embodiments, the vest includes hook and loops for adjusting fit.

In some embodiments, the system is optionally connected by wireless (for example Bluetooth) communications and/or wired communications to an application for optional control via computer, tablet, smartphone or the like.

In some embodiments, the application optionally includes visual and/or sound and/or verbal and/or sensible (e.g., vibration) feedback for the therapy and for device use.

In some embodiments, the feedback is used to guide the patient as to how to breathe, potentially enhancing treatment efficacy.

By way of a non-limiting example, there may be six independent pressure applicators (e.g., 6 inflatable chambers on a front side of the vest), positioned with respect to the lung lobes, designed to apply localized external pressure on the chest. In some embodiments, the pressure applicators may be independently activated according to a predefined inflation sequence.

It is noted that the number six is not intended to be limiting. Any integer number may be used, up to as large a number as desired for locating pressure on a patient's body. The number of inflatable sacs can be in a range between 1 and 40 or 100.

In some embodiments, the number of pressure applicators (e.g., inflatable chambers) may be even, and the inflatable chambers may be positioned symmetrically on a right and left side of a patient's upper body.

In some embodiments, the number of pressure applicators (e.g., inflatable chambers) may not be even, and the inflatable chambers may be positioned asymmetrically in relation to a right and left side of a patient's upper body.

In some embodiments, one or more of the pressure applicators (e.g., inflatable chambers) may be positioned to be placed centrally in relation to a right and left side of a patient's upper body. In some embodiments, one or more inflatable chambers may be positioned to be placed next to a patient's diaphragm and/or sternum and/or back.

In some embodiments, the vest may be stiff, providing a stiff surface against which the pressure applicators (e.g., inflatable sacs) can push, thereby applying pressure on a patient's torso. Furthermore, when the pressure applicators are deactivated (e.g., deflated), a quick release of compressed lungs is enabled.

In some embodiments, the vest may be flexible, providing a flexible surface against which the pressure applicators (e.g., inflatable sacs) can push, thereby applying pressure on a patient's torso.

In some embodiments, the vest may be flexible and stretchable, wherein the stretching may take up some pressure, still leaving some pressure for exertion upon the patient's torso.

Pressure Applicator Location(s)

In some embodiments, the pressure applicators are placed in pockets of the vest and/or integrated therewithin.

In some embodiments, a second, internal, flexible, possibly cloth-like vest is placed on a patient's torso. In some embodiments, the pockets are sewn in the second, internal vest, which is placed against a patient's torso, underneath the external vest. In some embodiments, the internal vest pockets determine the potential locations for the pressure applicators (e.g., inflatable sacs).

In some embodiments, the pressure applicators (e.g., inflatable sacs) optionally include one side of a hook-and-loop fastener, such as Velcro, and the vest includes another side of the hook-and-loop fastener, potentially enabling a physician/physiotherapist/technician flexibility in placing the inflatable sacs wherever the physician/physiotherapist/technician desires.

The pressure applicators (e.g., inflatable sacs) may be positioned:
to cover one lung lobe;
to cover each lung lobe;
to cover a lung lobe, optionally not the same number of sacs for each lung lobe,
at least one applicator (e.g., inflatable sacs) for each location where pressure is planned; and
optionally more than one applicator (e.g., inflatable sacs) for each location where pressure is planned, potentially enabling stacking two or more applicators pressing against one location.

In some embodiments, the number of the pressure applicators (e.g., inflatable sacs) are optionally derived from the anatomical location of the lung lobes.

In some embodiments, where the pressure applicators are inflatable sacs—the sacs are inflated by a gas, for example air.

In some embodiments, the gas does not exit the system when the sacs are deflated, but rather sucked into a gas chamber, optionally for reuse in a closed system.

In some embodiments, the sacs are inflated by a fluid, for example water or oil.

In some embodiments, when sacs are inflated by a fluid, the fluid does not exit the system when the sacs are deflated, but rather sucked into a fluid container, optionally for reuse in a closed system.

A potential advantage of example embodiments where the gas or fluid are used in a closed system can be that air from a patient's surroundings is not pumped into sacs, not potentially travelling to a different patient, not potentially released at the different, new patient's location. Such a system may potentially be suitable for use in an environment of contagious diseases, for example COVID-19.

Data Analysis

According to some embodiments, the system is integrated with sensors (e.g., pressure sensor sensing pressure of at least one of said pressure applicators, strain gauge, at least one microphone performing lung auscultation) for sensing at least one parameter associated with at least one physiological state (e.g., at least one stage within a breathing cycle of said patient, lung auscultation, coughing of said patient etc.) of said patient.

According to some embodiments, the system additionally includes feedback adapted to provide guidance, during said treatment, to instruct said patient as to a required breathing pattern.

In some embodiments, such guidance is based on said sensing of at least one of said physiological state of said patient.

According to some embodiments, the system is in communication with a communicable and readable database for collecting and storing such data from the sensor(s). The database optionally includes one or more parameters including: breathing patterns of a patient, trends in said breathing pattern, an eigenvector of said breathing patterns, a shape of at least a portion of said breathing pattern, an area under the at least a portion of said breathing pattern, any derivative of at least a portion of said breathing pattern, a number of coughs in treatment, a cough pattern, general compliance to a treatment protocol, a treatment's duration and the number of treatments the patient had in a predetermined period of time (e.g., week, month etc.).

In some embodiments, the database is analyzed, and said guidance (and/or treatment) is adjusted accordingly.

In some embodiments, the patient guidance (i.e., instruction how to breath) can be based on (a) the sensed parameter(s) of said patient, (b) a statistical (or any data analysis) analysis of collected data from a plurality of patients being stored in said database, and, (c) any combination thereof.

In some embodiments, Machine Learning, ML, and/or Artificial Intelligence, AI, algorithms are utilized.

The term “machine learning (ML)” or “artificial intelligence (AI)” refers hereinafter to the study of computer algorithms that improve automatically through experience and by the use of data. Machine learning algorithms build a model based on sample data, known as “training data”, in order to make predictions or decisions without being explicitly programmed to do so. Machine learning algorithms are generally used where it is difficult or unfeasible to develop conventional algorithms to perform the needed tasks.

Such ML and/or AI can be used to analyze the data collected and provide suggestions for better guidance of the patient for enhancing treatment efficacy.

According to some embodiments, by utilizing a Neural Network Analysis (NNA), which is based on machine-learning ability, the analysis of the database will continuously be improving with each additional performance as database grows. Thus, better, tailor made guidance (i.e., breathing instructions), could be provided. Such learning process (either by ML, AI or NNA) is optionally used to “educate” the data analysis to include more variations within its solution space.

According to some embodiments, the system as defined above, optionally includes 2 modes of operation: (a) a learning phase; and, (b) an operational phase.

One object of the present disclosure is to provide the system as defined above, wherein, in said learning phase, a machine learning model is trained to analyze at least one parameter (as described above) in the database, the treatment applied thereto and the clinical outcome, in order to generate information being indicative of said treatment and/or said guidance to provide enhance treatment.

In some embodiments, such information includes treatment protocols and corresponding clinical results obtained from such treatment protocols.

According to some embodiments, said data collected in said database is either supervised or unsupervised data.

According to some embodiments, in said operational phase, said system is adapted to provide suggestion and/or recommendation of different treatment protocols, based on said analysis.

Cleanable, Sterilizable

An aspect of some embodiments includes surfaces of components of the system being cleanable and/or sterilizable to medical standards.

In some embodiments, a replaceable and/or disposable outer vest cover is optionally used, to keep the vest clean, and replaced when replacement is warranted, and/or when transferred from one patient to another.

Programmed Timing of Inflation and/or Deflation

An aspect of some embodiments includes programming a specific order of application of pressure (e.g., inflating and/or deflating the inflatable sac or sacs).

In some embodiments, the timing of pressure application (e.g., inflation/deflation) is optionally synchronized with operation of a ventilator which ventilates a patient. In some embodiments, the timing thereof is optionally synchronized with natural breathing. In some embodiments, synchronization to breathing is optionally based on detection of inhalation/exhalation by a sensor, as described elsewhere herein.

In some embodiments, the application of pressure is maintained over more than one inspiration/expiration cycle of a patient, that lasts over more than one breathing cycle of the patient.

In some embodiments, a readiness and/or desire of a patient to cough is optionally detected, and timing of pressure application (e.g., inflation/deflation) is optionally synchronized with the detection.

In some embodiments, a patient is optionally assisted in coughing by timing of pressure application (e.g., inflation/deflation).

Console

In some embodiments, a console optionally includes the system's electronic module(s), control unit(s), power supply, an optional source for applying pressure (e.g., air compressor, pneumatic system), an optional display screen and operation buttons.

In some embodiments, when inflatable sacs are used, the pneumatic system includes an air compressor connected via tubing to a set of solenoid valves used to direct compressed air to a desired air pocket.

In some embodiments, a pressure sensor is used to regulate the pressure within the chambers and a pressure release valve is optionally used to prevent pressure overload.

Smart, Adaptive

An aspect of some embodiments includes programming a controller to change an order of activation of the pressure applicators (e.g., inflating and/or deflating the inflatable sac or sacs).

In some embodiments, the change in program is optionally entered by a physician.

In some embodiments, the change in program is optionally based on measurement of physiological parameters associated with measuring and analyzing a patient's breathing.

In some embodiments, locations for placing the pressure applicators (e.g., inflatable sacs) are optionally generated automatically based on image analysis of an image of a patient's lungs. By way of a non-limiting example, a pressure applicator (e.g., inflatable sac) may optionally be placed over a lung lobe that shows evidence of secretions, potentially to exert pressure on that lung lobe. In some embodiments, the same can be placed over a lung lobe that shows evidence that the lung lobe is clear, or relatively clear, potentially to exert pressure on that lung lobe.

In some embodiments, a program for inflating the inflatable sacs is optionally generated automatically based on image analysis of an image of a patient's lungs.

Closing the Device's Control Loop

An aspect of some embodiments includes collecting sensor measurements, and determining a program of treatment based on the measurement.

An aspect of some embodiments includes collecting sensor measurements, and activating vest operation based on the measurement.

In some embodiments, more than one type of sensor is used, and input from the more than one sensor may optionally be combined to determine the program of treatment.

In some embodiments, the sensors are one or more microphones, optionally inserted into the vest, or even attached to the vest. The microphones may perform auscultation, detecting lung parameters such as air ventilation, or, if there is no air ventilation, wet crackles, dry crackles, fine crackles, secretion transport, wheezing, and cough.

In some embodiments sensing and/or testing for breathing is optionally done by using a dedicated sac or pillow in the vest.

In some embodiments, the sensing is by using a pressure sensor sensing the dedicated sac/pillow. In some embodiments, the dedicated sac may be inflated at different times than other sacs, in order to enable pressure sensing unrelated to using other sacs to exert pressure on the patient's lungs. In some embodiments, the dedicated sac may be inflated before other sacs. In some embodiments, the dedicated sac may not be deflated even when other sacs are deflated.

In some embodiments, the sensing is by using a microphone sensing at the dedicated sac/pillow.

In some embodiments sensing and/or testing for breathing is optionally done by using a strap or belt around a patient's chest, which includes a strain gauge. The strain gauge provides a signal which is optionally used to estimate changes in chest circumference related to breathing and/or related to pressure applied between the straps and the chest.

In some embodiments, the sensor(s) are one or more volumeters, potentially enabling to estimate lung inflation and/or deflation and/or lung compliance. In some embodiments, the sensor(s) that can measure volume of adjacent space. Such a sensor potentially enables measuring lung inflation and/or deflation.

In some embodiments, the sensor(s) are one or more pressure sensors.

In some embodiments, the sensor(s) are one or more impedance sensors.

In some embodiments, the sensor(s) are optionally arranged symmetrically relative to the vest.

In some embodiments, the sensors are one or more imaging system, which optionally provide one or more images of a patent's lungs, which are optionally used to determine the locations of the pressure applicators (e.g., inflatable sacs), and/or which should be activated (e.g., inflatable sacs to inflate/deflate).

Target Population

A non-limiting extent of a population for which example embodiments can potentially be used includes patients with Cystic Fibrosis (CF), COPD, Bronchiectasis, asthma and lung diseases with secretory problems, patients with neuromuscular disease affecting an ability to effectively cough.

Clearing a Patient's Airways or Ventilating the Patient or Both

The above description of “closing the loop” optionally relates to performing treatment for clearing a patient's airways based on sensor measurement and/or image processing, and optionally relates to combining clearing the patient's airways and ventilating the patient.

Negative Pressure

An aspect of some embodiments includes potentially producing a state of negative pressure in a patient's lungs. In some embodiments, pressure applied by the pressure applicators (e.g., inflatable sacs) is released rapidly, by way of a non-limiting example in less than 0.5 second, or in less than 0.25 second. The patient's torso potentially rebounds, producing a rapid lowering of pressure in the patient's lungs, or at least in a location where the pressure applicators (e.g., inflatable sacs) are deactivated. The lowering of pressure may optionally induce air to enter the patient's lungs.

In some embodiments, a system as described herein potentially improves mucus transport and potentially improves efficacy of suction.

In some embodiments, a system as described herein potentially assists devices that produce negative pressure.

In some embodiments, a system as described herein potentially assists a person using a positive expiratory pressure (PEP) device. PEP devices optionally allow air to flow freely as a patient breathes in, but not when the patient breathes out. The patient breathes out harder against the resistance. In some embodiments, a vest assists a patient to breathe out by pressing on the patient's body.

A more detailed description is now provided, of a non-limiting example of some embodiments.

In some embodiments, a pneumatic vest is positioned on a patient's torso, having one or more of the following characteristics
a. The vest may optionally have a firm envelope.
b. at least one pressure applicator (e.g., inflatable sacs), optionally 2, 3, 4, 5, 6, 7, 8, 9, 10, and on up to 40 or even 50.
c. The system potentially raises thoracic resistance in hyper-inflated areas.
d. The system potentially direct air flow to closed peripheral areas of the lung.
e. The system potentially increases an effective volume of the lung by potentially increasing air flow in the small airways.
f. The system potentially enables secretion clearance by delivering the secretion to larger airways.
g. The system is optionally synchronized with a ventilation machine.

In some embodiments, the system includes one or more sensors, as described elsewhere herein.

In some embodiments, the system includes a controller, as described elsewhere herein.

In some embodiments, the controller is configured to provide feedback and/or guidance to a patient and/or caregiver, as described elsewhere herein.

In some embodiments, at least two pressure applicators overlap in their position so as to better apply the pressure on the patient's torso.

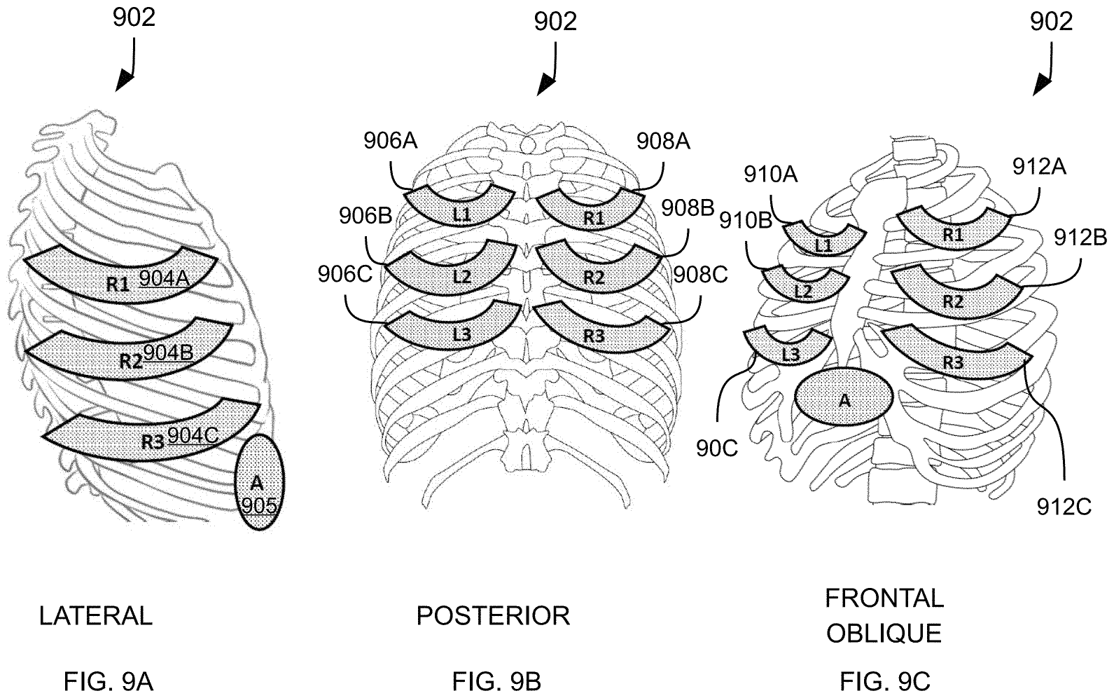

In some embodiments, by way of a non-limiting example as shown in FIGS. 9A-9C, a first pair of inflatable sacs is U shaped and covers the upper lobe area, a second pair of inflatable sacs is placed just inferior to the first pair, and is also U shaped, as is a third pair.

In some embodiments, a first pair is against a lower portion of the patient's torso, a second pair is located higher, above the first pair along the torso toward the patient's head, and the third pair is located still higher, above the second pair, still further along the torso toward the patient's head.

In some embodiments, a controller potentially:

controls activation and/or deactivation of the pressure applicators; in case of inflatable sacs—inflation and/or deflation of the inflatable sacs, optionally controlling inflation and deflation of each one of the inflatable sacs separately;

operates in synchronization with a ventilator;

controls the inflation to prevent high pressure from potentially causing damage to the lungs, optionally by measuring inflation pressure of the inflatable sacs; and

includes an algorithm for monitoring the system.

In various embodiments the vest operates in conjunction with a device other than a ventilator, for example a BiPAP device and/or a cough stimulating device.

In various embodiments the vest operates in conjunction with a positive pressure device, for example such as a BiPAP device, and/or with a negative pressure device.

An example limit of pressure per inflatable sac is optionally approximately 50-150 millibars. The controller optionally controls and prevents pressure higher than the limit;

In some embodiments, the system is optionally interfaced with a ventilation system. In some embodiments, a connection is made to a ventilation pipe of a ventilation machine. In some embodiments, the connection is optionally configured not to transfer air from the ventilation pipe.

In some embodiments, the system is packaged as a sealed package which can be cleaned and/or sterilized.

In some embodiments, the system produces no more than 60 dB noise at a distance of 0.5 meters.

In some embodiments, the system is independent of power, for example powered by batteries.

In some embodiments, the system uses environmental air for inflation, for example local room air, optionally unfiltered.

In some embodiments, a portion of the system placed on the patient weighs no more than 8 kilograms.

Some features of an example embodiment vest include:

a. The vest is optionally easy to put on and remove from/by a patient;

b. The vest is optionally comfortable against a patient's skin, optionally put directly against a patient's skin;

c. The vest may be closed by various closing methods, such as a zipper, straps, hook-and-loop and buckles. In some embodiments the closing is adjustable, such as by using cinches and/or straps and/or buckles, and/or hook-and-loop (Velcro) closing;

d. The vest is configured so as not to shift unduly on a patient's torso after closing, that is, not shift up and down more than 1-3 centimeters, and/or not to shift side-to-side more than 1-3 centimeters;

e. The vest is optionally configured not to expand under pressure, so as to direct the pressure toward the patient;

f. Each of the pressure applicators (e.g., inflatable sacs) is independently activated; or several (or all) pressure applicators are activated simultaneously;

g. The vest is optionally lightweight, for carrying by hand from patient to patient;

h. The vest is made of a material which is sterilizable by medical grade sterilization, for example concentrated alcohol solutions.

Some features of an example embodiment of a controller include:
a. Releases pressure from the inflatable sacs during 0.25 to 0.5 seconds.
b. The controller optionally includes indications or warnings of one or more of:
power on/off;
high pressure;
pressure of each pressure applicator (e.g., inflatable sac);
low pressure per pressure applicator (e.g., inflatable sac);
deactivation (e.g., deflation) status per pressure applicator (e.g., inflatable sac);
operation program selected; and
time and/or date.

In some embodiments releasing pressure from the inflatable sacs is to a lower pressure, not necessarily to zero positive pressure.

In some embodiments, releasing the pressure is by opening a valve which enables release of the pressure. The pressure may not drop to ambient pressure, or to zero, but drops in relation to pressure previously present and exerted on the patient's upper body.

In some embodiments, the controller will perform a system test upon initial operation.

In some embodiments, the controller optionally deactivates the pressure applicators (e.g., inflatable sacs=deflates the sacs) when the system determines that a patient is coughing or is to cough.

In some embodiments, the controller optionally detects a patient starting or preparing to cough by measuring pressures, either in the ventilation system, or in the inflatable sacs, caused by the coughing or by the preparation for the cough.

In some embodiments, the controller is optionally calibrated to detect a cough, setting pressure levels and or durations, per patient.

In some embodiments, the controller is optionally calibrated to set a duration of deflation for a cough, optionally setting the duration and/or rate of deflation per patient.

In some embodiments, a record of inflation and deflation times is optionally kept.

In some embodiments, inflation and deflation of the sacs is optionally synchronized with a ventilator.

In various embodiments the vest operates in conjunction with a non-invasive ventilator, for example a BiPAP device and/or a cough simulating device.

In various embodiments the vest operates in conjunction with an invasive ventilator.

In various embodiments the vest operates in conjunction with a patient's natural breathing, without involving a ventilation device.

In some embodiments, a user interface enables setting and/or selecting pressure activation/deactivation (e.g., inflation/deflation) programs (such as sequences and/or durations per sac).

In some embodiments, the controller operates a program which gradually inflates all the inflatable sacs over 3-4 respiratory cycles, and deflates all the inflatable sacs at once.

In some embodiments, a system as described herein is based on Autogenic Drainage (AD) airway clearance therapy. Such a therapy method aims to produce expiratory airflow in different generations of the bronchi.

In some embodiments, the therapy aims to produce the expiratory airflow simultaneously with an active, but not forced expiration.

By breathing at lower lung volumes, secretions are systematically transported from peripheral to more central airways, where an effective cough can expectorate them.

Typically, AD includes three breathing phases—unstick, collect and evacuate.

In the unstick phase, small breaths at low volumes are taken, which affect the peripheral airways of the chest. This slow and deep air movement potentially loosens peripheral secretions.

In the collect phase, medium-sized breaths are performed, which affect the more proximal airways of the chest, and secretions are potentially collected from central airways by breathing at low to middle lung volumes.

In the evacuate phase, full breaths at mid to high lung volumes are taken, expelling secretions from the central airways. In some embodiments, the breaths are slow breaths. Adjusting the depth and location of lung volumes during respiration potentially generates shearing forces induced by airflow, which loosen, mobilize, and move secretions from the peripheral to the central airways, from which they can be expelled by coughing.

In some embodiments, the vest's inflation sequence is designed to emulate the breathing phases of the AD airway clearance therapy. The vest gradually applies localized external pressure on the chest to promote breathing at different lung volumes and at different areas of the lungs.

In some embodiments, at first, the vest's uppermost air chambers are inflated, followed by the next levels of chambers, in order and so on.

Next, inflation of all chambers is performed, for the ‘unstick’ phase. In some embodiments, the patient is instructed, at this time, to breathe at low lung volumes.

Next, the bottom row of chambers is deflated and inflated alternately for several cycles, transitioning from the unstick to the collect phase.

Once completed, all vest chambers are deflated simultaneously, enabling the ‘evacuate’ phase.

The above sequence may be repeated.

In some embodiments, activation of the vest is combined with Positive Airway Pressure (PAP), provided by a ventilator, potentially enabling air to ventilate more distal areas in the bronchial tree.

In some embodiments, Positive Expiratory Pressure assists the patient in balancing expiratory forces during treatment, resulting in longer and deeper expiration and potentially preventing the collapse of smaller airways.

Reference is now made to FIG. 2D, which is a simplified graph illustration of Autogenic Drainage (AD) airway clearance therapy according to an example embodiment.

FIG. 2D shows a graph 220, having an X-axis 224 of time, and a Y-axis 222 of breathing volume.

FIG. 2D shows one example of an AD treatment cycle, including several breathing cycles.

FIG. 2D shows stage 1 254—a low volume breathing stage to mobilize secretions from peripheral airways, qualitatively corresponding to the unstick phase in the AD treatment cycle; stage 2 256—a medium or tidal volume breathing stage to collect mucus from middle airways, qualitatively corresponding to the collect phase in the AD treatment cycle; and stage 3—a larger volume breathing stage to enable expectoration from central airways, qualitatively corresponding to the evacuate phase in the AD treatment cycle.

FIG. 2D shows a line 232 of air volume in a lung of a patient, over time.

Stage 1 254 includes several cycles of breathing at low volume, followed by stage 2 256 including several cycles of breathing at higher volume, followed by stage 3 258 including several cycles of breathing at even higher volume.

In some embodiments, stage 3 is optionally followed by a sudden deflation (not shown in FIG. 2D).

FIG. 2D shows the line 232 in relation to several physiological features typically associated with a patient's lungs: TLC (Total Lung Capacity) 242; FRC (Functional Residual Capacity) 244; TV (Tidal Volume) 243; and RV (Residual Volume) 245.

In some embodiments, pressure applicators (e.g., inflatable sacs) are activated and deactivated by synchronization with a patient's natural exhalation and inhalation, respectively. The patient's breathing cycle is optionally tracked, and transition between the stages is optionally activated according to a ‘Cycle Interval’ setting.

By way of one non-limiting example, with a ‘Cycle Interval’ setting of 3 cycles, after reaching full inflation of the top chambers, upon completion of 3 breathing cycles a consecutive level of chambers is inflated when the user exhales.

In some embodiments, when a cough session is detected the pressure applicators (e.g., inflatable sacs) within the vest are deactivated (e.g., deflated) automatically. In some embodiments, may be by receiving an indication from a patient that the patient wants to cough. In some embodiments, detecting a cough section may be done automatically, for example by monitoring the pressure and detecting a change from a regular breathing pattern, or noise on the regular breathing monitoring signal.

According to some embodiments, at least one microphone is utilized to perform lung auscultation, such that detection of air ventilation, wet crackles, dry crackles, fine crackles, secretion transport, wheezing, cough and any combination thereof is enabled.

In some embodiments, the pressure applicators (e.g., inflatable sacs) may be activated (i.e., inflated) while the patient exhales.

In some embodiments, the pressure applicators (e.g., inflatable sacs) may be deactivated (i.e., deflated) while the patient inhales.

Reference is now made to FIG. 2E which is a simplified graph illustration of application of pressure by chambers of a vest synchronized with a breathing cycle, according to an example embodiment.

FIG. 2E shows a graph 260, having an X-axis 262 of time and a Y-axis 261 of pressure.

The graph 260 shows a line 263 describing pressure in a patient's lungs over time, which can serve as an indication of a breathing pattern. References 268 and 267 refer to exhalation and inhalation, respectively.

The graph 260 shows one example of a treatment cycle, including several breathing cycles with application and removal of pressure (e.g., by inflating and deflating inflatable sacs).

The graph 260 shows an optional initial time period 264 during which a patient's breathing is monitored and/or detected.

In some embodiments, the patient's breathing is monitored by a microphone sensor.

In some embodiments, the patient's breathing is monitored by a pressure sensor. In some embodiments, where the patient's breathing is monitored by one of the pressure applicators (e.g., inflatable sacs). For example, one or more inflatable sacs are inflated, or the one or more inflatable sacs are already inflated, so that pressure of the patient's lungs can be monitored by measuring an associated pressure in the inflated sac(s) which are located against the patient's upper body.

FIG. 2E shows an additional time period 265 during which the pressure applicators (e.g., inflatable sacs) are activated (e.g., inflated) for providing an AD treatment cycle.

During the additional time period 265 inflatable sacs located against a top portion of the patient's upper body are inflated 269A, and at an end of the additional time period 265 the inflatable sacs located against a top portion of the patient's upper body are deflated 269D.

During another time period 266 inflatable sacs located against a bottom portion of the patient's upper body are inflated 269B, and at an end of the other time period 266 the inflatable sacs located against the bottom portion of the patient's upper body are deflated 269C.

In some embodiments, inflation and deflation of the vest air sacs is optionally synchronized with a patient's natural exhalation and inhalation, respectively. The patient's breathing cycle is optionally tracked, and transition between the stages is optionally activated according to a ‘Cycle Interval’ setting.

In some embodiments, the pressure applicators (e.g. inflatable sacs) are activated (e.g. inflated) while the patient exhales.

In some embodiments, the pressure applicators (e.g. inflatable sacs) may be deactivated (deflated) while the patient inhales.

In some embodiments, when a cough session is detected the vest deflates automatically.

Reference is now made to FIG. 2F, which is a simplified flow chart illustration of a treatment cycle according to an example embodiment.

FIG. 2F shows a process which includes:

initiating treatment (272);

applying pressure (e.g. inflating) using the pressure applicators (e.g., inflatable sacs) positioned at the top (274);

applying pressure (e.g. inflating) using the pressure applicators (e.g., inflatable sacs) positioned at the bottom (276);

releasing pressure (e.g. deflating) applied by the pressure applicators (e.g., inflatable sacs) positioned at the bottom (278);

optionally repeating the activating (e.g. inflating) the pressure applicators (e.g., inflatable sacs) positioned on the bottom (276) and deactivating (e.g. deflating) the pressure applicators (e.g., inflatable sacs) positioned on the bottom (278) for N cycles (280); and

releasing all pressure (282).

In some embodiments, releasing all pressure (282) includes releasing all pressure except for pressure in one or more pressure applicators which may be used for sensing.

Reference is now made to FIG. 2G, which is a simplified flow chart illustration of a method of treatment according to an example embodiment.

FIG. 2G shows a process which includes:

initiating treatment (290);

inflating one or more monitoring chamber(s) (291). In some embodiments, one or more chambers are used to monitor the patient's lungs and/or breathing, upon a vest, and the one or more monitoring chamber(s) may optionally be inflated before other chambers;

inflating top chambers (292);

inflating bottom chambers (293);

deflating bottom chambers (294);

optionally repeating the inflating bottom chambers (293) and deflating bottom chambers (294) for N cycles (295); and

deflating all chambers (296).

In some embodiments, deflating all chambers (296) includes releasing all pressure except for pressure in one or more chambers which may be used for sensing.

In some embodiments, after inflating the one or more monitoring chamber(s) (291) a patient's breathing is monitored.

In some embodiments, pressure in the one or more monitoring chamber(s) may be monitored. In some embodiments, a pressure signal is optionally analyzed to detect the patient's breathing cycle. The pressure signal may be used to detect where during a breathing cycle the patient is at a certain time.

In some embodiments, a microphone next to the one or more monitoring chamber(s) may be monitored. In some embodiments, sound is optionally analyzed to detect the patient's breathing cycle. The sound may be used to detect where during a breathing cycle the patient is at a certain time.

In some embodiments, the system optionally controls inflation and deflation based on where during a breathing cycle the patient is at a certain time.

In some embodiments, the system optionally provides guidance, optionally based on the monitoring, to the patient as to how to breathe, thereby enabling the patient to cooperate with and/or assist the treatment being provided.

The guidance may be in form of one or more of displaying written instructions; providing voiced instructions; providing sound instructions; providing sensory instructions such as vibrations and displaying visual indications for a desired type of breathing.

In some embodiments, the monitoring may be used to detect coughing. In some embodiments, when coughing is detected, the inflatable sacs are deflated.

An Example Algorithm Description

In some embodiments, the system tracks pressure in one or more a designated air chamber(s) by a pressure sensor. Chest movement while breathing results in pressure changes in the chamber, and the user's breathing pattern is detected based on the sampled pressure signal. Detection of the breathing cycle potentially enables to synchronize the inflation and deflation of the vest chambers with the exhalation and inhalation of the patient respectively.

Reference is now made to FIG. 2H, which is a simplified flow chart illustration of a method of treatment according to an example embodiment.

The method of FIG. 2H includes:

inflating one or more inflatable sacs located next to a top of the patient's torso (284);

inflating one or more inflatable sacs located next to a bottom of the patient's torso (285);

deflating the one or more inflatable sacs located next to the bottom of the patient's torso (286);

repeating the inflating and deflating the one or more inflatable sacs located next to the bottom of the patient's torso a plurality of times (287); and

deflating all the inflatable sacs (288).

Reference is now made to FIG. 2I, which is a simplified flow chart illustration of a method of treatment according to an example embodiment.

The method of FIG. 2I includes:

sensing a signal associated with a patient's breathing (342);

analyzing the signal (344); and

providing guidance to the patient to control the patient's breathing (346).

Reference is now made to FIG. 2J, which is a simplified flow chart illustration of a method of treatment according to an example embodiment.

The method of FIG. 2J includes:

sensing a signal associated with a patient's breathing (352);

analyzing the signal (354); and

activating or deactivating pressure applicators based on the analysis (356).

In some embodiments, the activating pressure applicators includes changing an amount of pressure exerted on the patient's lungs.

In some embodiments, the activating pressure applicators includes changing the location of pressure exerted on the patient's lungs, optionally by controlling which pressure applicators are activated.

In some embodiments, the changing the location and/or amount of pressure exerted on the patient's lungs includes changing inflating and/or deflation of one or more inflatable sacs positioned to exert pressure on the patient's lungs and/or abdomen.

Reference is now made to FIG. 2K, which is a simplified flow chart illustration of a method of treatment according to an example embodiment.

The method of FIG. 2K includes:

activating or deactivating pressure applicators on a patient's torso (362);

sensing and analyzing a signal associated with the patient's breathing (364); and

optionally providing guidance to the patient to control the patient's breathing (368).

In some embodiments, the sensing and analyzing includes controlling 366 the activating or deactivating the pressure applicators on the patient's torso (362).

In some embodiments, the method of FIG. 2K also includes changing the location and/or amount of pressure exerted on the patient's lungs.

In some embodiments, the changing the location and/or amount of pressure exerted on the patient's lungs includes changing inflating and/or deflation of one or more inflatable sacs positioned to exert pressure on the patient's lungs and/or abdomen.

Example User Interface

In some embodiments, a display screen and/or operation buttons on a console are used to adjust therapy settings and manage device operation.

In some embodiments, the display screen and/or operation buttons are optionally on a console packaged with the air compressor. In some embodiments, the display screen and/or operation buttons are optionally on a console attached to the vest.

In some embodiments, the display screen and/or operation buttons are optionally on a smart phone or tablet or other type of mobile computing device.

A patient or a caregiver can select required pressure level, therapy duration, and treatment protocol to adhere to a prescribed therapy ordered by a physician.

In some embodiments, during therapy, the patient can press for a ‘Cough Pause’, where the vest deflates and enables the user to cough in order to clear secretions.

In some embodiments, a user interface may include patient breathing guidance as one or more of displaying written instructions; providing voiced instructions; providing audio instructions; and displaying visual indications for a desired type of breathing.

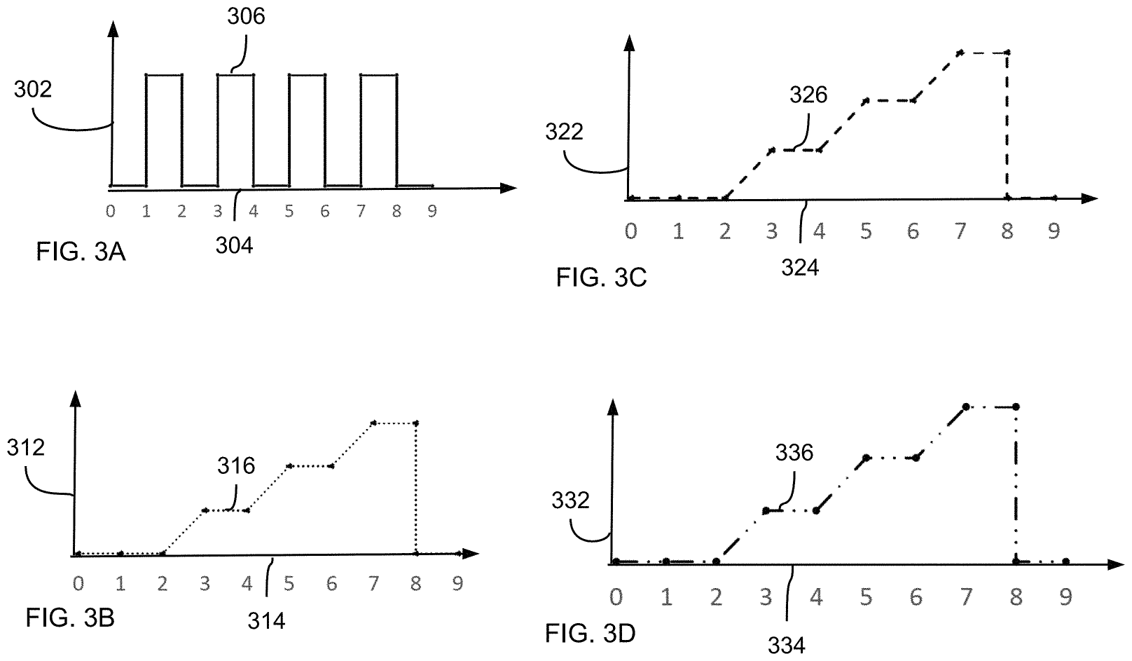

Reference is now made to FIGS. 3A-3D, which are simplified graphical illustrations of respiration and pressure at three pairs of inflatable sacs according to an example embodiment.

FIGS. 3A-3D are all graphs, with x-axes 304 314 324 334 showing qualitative time, where a unit of the x-axis 304 stands for inspiration or expiration and Y-axes 302 312 322 332 showing qualitative pressure, without units.

FIGS. 3A-3D show one example program of inflating inflatable sacs optionally synchronized with a ventilating machine.

FIG. 3A shows a first line 306, indicating pressure of air in a ventilating machine.

FIGS. 3B-3D shows lines indicating pressure provided to inflatable sacs.

FIG. 3B shows a second line 316 indicating pressure provided to a first pair of inflatable sacs.

FIG. 3C shows a third line 326 indicating pressure provided to a second pair of inflatable sacs.

FIG. 3D shows a fourth line 336 indicating pressure provided to a third pair of inflatable sacs.

FIGS. 3A-3D show the example program gradually inflating the inflatable sacs over several (3-4) respiratory cycles of the ventilating machine. It should be noted that the above disclosure referred to inflatable sacs; however, in some embodiments, it can relate to electrically activated mechanical pads or any other pressure applications means.

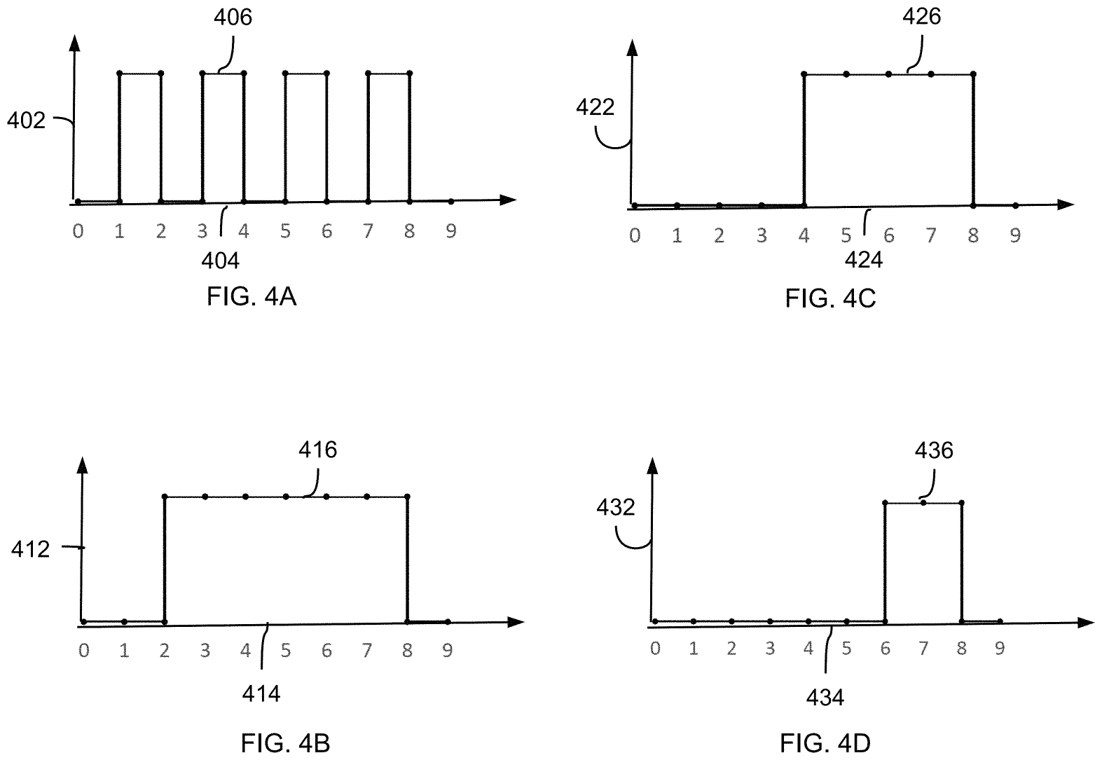

Reference is now made to FIGS. 4A-4D, which are simplified graphical illustrations of respiration and pressure at three pairs of inflatable sacs according to an example embodiment.

FIGS. 4A-4D are graphs, with x-axes 404 414 424 434 showing qualitative time and Y-axes 402 412 422 432 showing qualitative pressure.

The description of the Figures relates to inflatable sacs; however, it can also relate to electrically activated mechanical pads or any other pressure applications means.

FIGS. 4A-4D show one example program of inflating inflatable sacs optionally synchronized with a ventilating machine.

FIG. 4A shows a first line 406, indicating pressure of air in a ventilating machine.

FIGS. 4B-4D shows lines indicating pressure provided to inflatable sacs.

FIG. 4B shows a second line 416 indicating pressure provided to a first pair of inflatable sacs.

FIG. 4C shows a third line 426 indicating pressure provided to a second pair of inflatable sacs.

FIG. 4D shows a fourth line 436 indicating pressure provided to a third pair of inflatable sacs.

FIGS. 4A-4D show the example program gradually inflating the inflatable sacs, first one pair of sacs, then a second pair of sacs, then a third pair of sacs, over several (3-4) respiratory cycles of the ventilating machine.

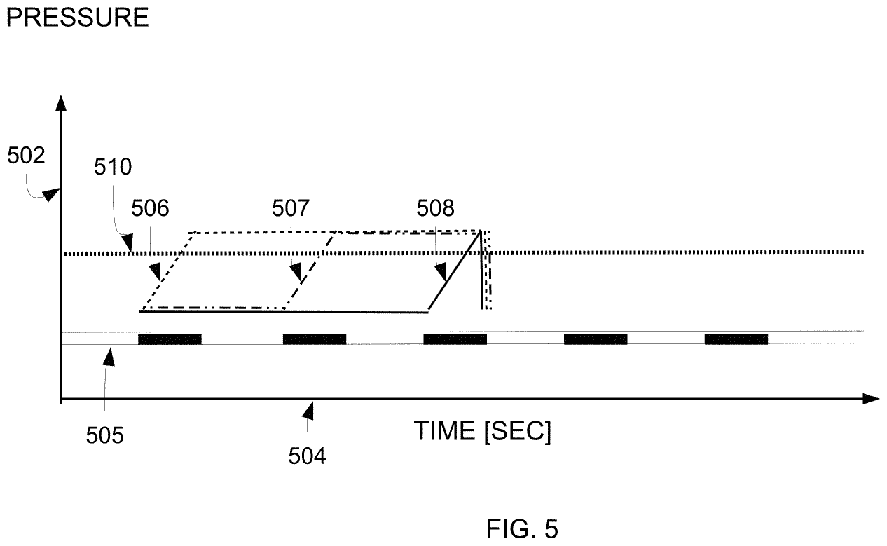

Reference is now made to FIG. 5, which is a simplified graphical illustration of respiration and pressure at three inflatable sacs according to an example embodiment.

The description of FIG. 5 relates to inflatable sacs; however, it can also relate to electrically activated mechanical pads or any other pressure applications means.

FIG. 5 is a graph, with an x-axis 504 showing qualitative time and a Y-axis 502 showing qualitative pressure.

FIG. 5 shows one example program of inflating inflatable sacs optionally synchronized with a ventilating machine.

FIG. 5 shows a first horizontal bar 505, with white sections indicating inspirium and dark sections indicating expirium.

FIG. 5 shows lines indicating pressure provided to inflatable sacs.

FIG. 5 shows a first line 506 indicating pressure provided to a first inflatable sac, a second line 507 indicating pressure provided to a second inflatable sac, and a third line 508 indicating pressure provided to a third inflatable sac.

FIG. 5 also shows a fourth line 510, indicating PIP (Positive Inspiratory Pressure).

FIG. 5 shows that the pressure provided to the inflatable sacs increases to above the PIP pressure. By way of a non-limiting example, the pressure provided is approximately in a range of 20-40 cm H₂O.

In some embodiments, a lung may be healthy, or heathier, on one side, and sick, or more sick, on another side.

Table 1 below describes a program, or method, or algorithm of treatment of a patient's lungs. Table 1 shows timing of activation and deactivation (e.g., inflation and deflation) of the pressure applicators (e.g., inflatable sacs) in relation to the patient's breathing cycle. Table 1 shows “+” marking a pressure activation of the pressure applicators (e.g., inflatable sacs being in the inflated state), “−” marking a pressure deactivation of the pressure applicators (e.g., inflatable sacs being in the non-inflated state). The columns of Table 1 indicate inspirium (Ins) and expirium (Exp) cycles, and the rows of Table 1 indicate a state of one of three inflatable sacs “H1”, “H2” and “H3” on a healthy or more-healthy side of the lungs, and three inflatable sacs “S1”, “S2” and “S3” on a sick or more-sick side of the lungs. The method of Table 1 is called herein the “Healthy Sick” algorithm.

TABLE 1

| | | | | | | | | | | | | New |
|---|---|---|---|---|---|---|---|---|---|---|---|---|
| | Ins1 | Exp1 | Ins2 | Exp2 | Ins3 | Exp3 | Ins4 | Exp4 | Ins5 | Exp5 | Ins6 | cycle |
| H1 | − | + | + | + | + | + | + | + | + | + | − | + |
| H2 | − | − | − | + | + | + | + | + | + | + | − | − |
| H3 | − | − | − | − | − | + | − | − | − | + | − | − |
| S1 | − | + | + | + | + | + | − | + | + | + | − | + |
| S2 | − | − | − | + | + | + | − | − | − | + | − | − |
| S3 | − | − | − | − | − | + | − | − | − | + | − | − |

Table 2 below describes a program, or method, or algorithm of treatment of a patient's lungs, with “+” marking pressure activation of the pressure applicators (e.g., inflatable sacs being in the inflated state), “++” marking more-pressure being applied state (i.e., a more inflated state), “+++” marking a yet-more pressure being applied state (i.e., a more inflated state), and “−” marking a pressure deactivation of the pressure applicators (e.g., pressure released from the inflatable sacs). The columns of Table 2 indicate inspirium and expirium cycles, and the rows of Table 2 indicate a state of one of three right-side inflatable sacs “R1”, “R2” and “R3”, and three left-side inflatable sacs “L1”, “L2” and “L3”. The method of Table 2 is called herein “gradual compression shift”.

TABLE 2

| | | | | | | | | | | | | New |
|---|---|---|---|---|---|---|---|---|---|---|---|---|
| | Ins1 | Exp1 | Ins2 | Exp2 | Ins3 | Exp3 | Ins4 | Exp4 | Ins5 | Exp5 | Ins6 | cycle |
| R1 | − | + | + | ++ | ++ | +++ | ++ | ++ | + | + | − | + |
| R2 | − | + | + | ++ | ++ | +++ | ++ | ++ | + | + | − | + |
| R3 | − | + | + | ++ | ++ | +++ | ++ | ++ | + | + | − | + |
| L1 | − | + | + | ++ | ++ | +++ | ++ | ++ | + | + | − | + |
| L2 | − | + | + | ++ | ++ | +++ | ++ | ++ | + | + | − | + |
| L3 | − | + | + | ++ | ++ | +++ | ++ | ++ | + | + | − | + |

Table 3 below describes a program, or method, or algorithm of treatment of a patient's lungs, with “+” marking an inflated state of an inflatable sac, “++” marking a more-inflated state, “+++” marking a yet-more inflated state, and “−” marking a non-inflated state of the inflatable sac. The columns of Table 3 indicate inspirium and expirium cycles, and the rows of Table 3 indicate a state of one of three right-side inflatable sacs “R1”, “R2” and “R3”, and three left-side inflatable sacs “L1”, “L2” and “L3”. The method of Table 3 is called herein “Lower Lobes Ventilation”.

TABLE 3

| | | | | | | | | | | | | New |
|---|---|---|---|---|---|---|---|---|---|---|---|---|
| | Ins1 | Exp1 | Ins2 | Exp2 | Ins3 | Exp3 | Ins4 | Exp4 | Ins5 | Exp5 | Ins6 | cycle |
| R1 | − | + | + | ++ | ++ | +++ | ++ | ++ | + | + | − | + |
| R2 | − | + | + | ++ | ++ | +++ | ++ | ++ | + | + | − | + |
| R3 | − | − | − | − | − | − | − | − | − | − | − | − |
| L1 | − | + | + | ++ | ++ | +++ | ++ | ++ | + | + | − | + |
| L2 | − | + | + | ++ | ++ | +++ | ++ | ++ | + | + | − | + |
| L3 | − | − | − | − | − | − | − | − | − | − | − | − |

In some embodiments, each “+” in the above tables optionally stands for a pressure of approximately 5-15 cm H₂O.

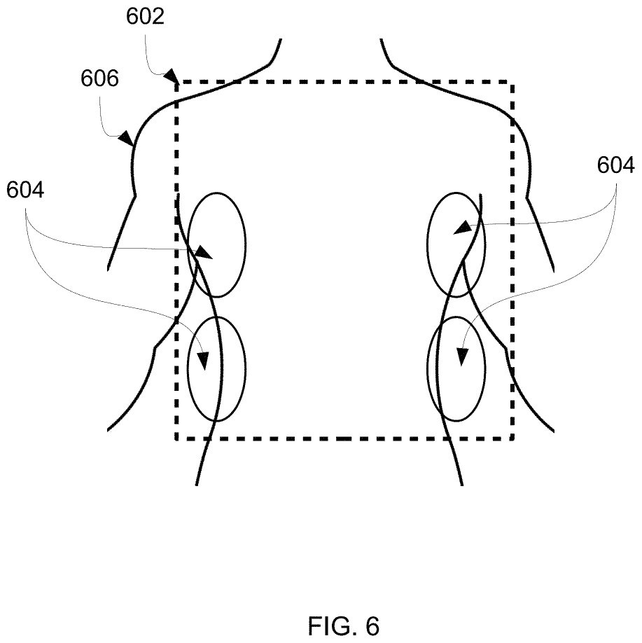

Reference is now made to FIG. 6, which is a simplified schematic illustration of a system constructed according to an example embodiment.

FIG. 6 shows a basic embodiment, including a vest 602 and pressure applicators (e.g., inflatable sacs) 604, placed qualitatively on a drawing of a patient's torso 606.

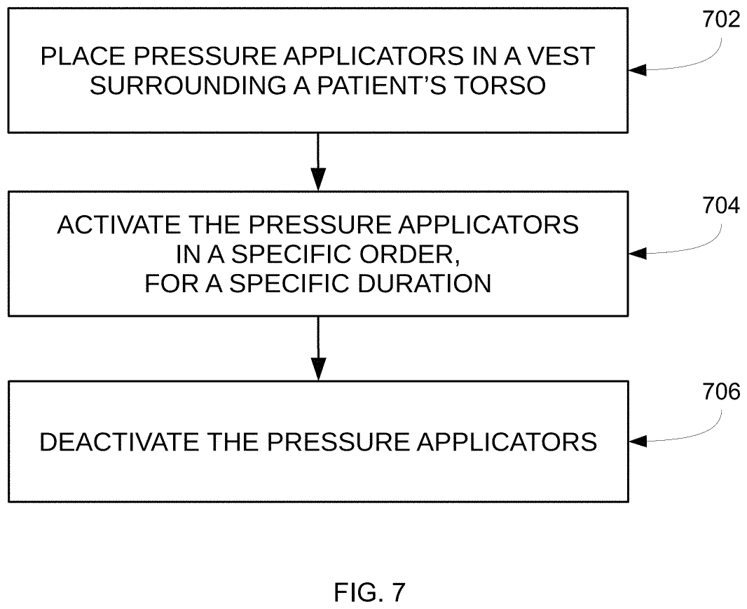

Reference is now made to FIG. 7, which is a simplified flow chart illustration of a method for clearing lung airways according to an example embodiment.

FIG. 7 shows a flow chart of a method of a basic embodiment.

The method of FIG. 7 includes:

placing the pressure applicators (e.g., inflatable sacs) in a vest surrounding a patient's torso; (702)

activating (e.g., inflating) the pressure applicators (e.g., inflatable sacs) in a specific order, for a specific duration (704); and

deactivating (e.g., deflating) the pressure applicators (e.g., inflatable sacs) (706).

Additional locations and in additional arrangements for inflatable sacs are now described.

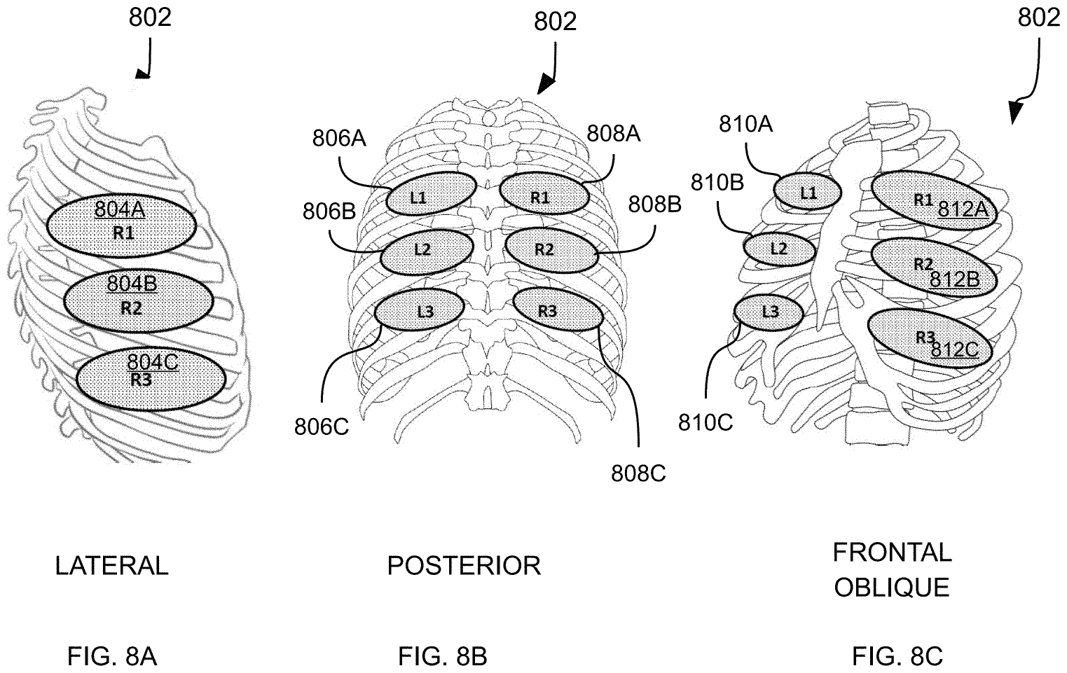

Reference is now made to FIGS. 8A-8C, which are simplified illustrations of one or more inflatable sacs in relation to a patient, in a system constructed and operational according to an example embodiment.

FIGS. 8A-8C, show non-limiting examples of a patient and the pressure applicators (e.g., inflatable sacs).

FIGS. 8A-8C do not show the vest (such as shown in FIG. 1), so that the vest does not obscure a view of the sacs.

FIG. 8A shows a lateral view of a skeleton 802 representing a patient, and inflatable sacs R1 804A R2 804B R3 804C placed on example locations on a right side of the patient. The markings R1 R2 R3 are used to indicate example locations on a right side of the patient.

FIG. 8B shows a posterior view of the skeleton 802 representing a patient, and inflatable sac locations on the back of the patient, L1 806A L2 806B L3 806C on a left side of the back of the patient and R1 808A R2 808B R3 808C on a right side of the back of the patient.

FIG. 8C shows a frontal oblique view of the skeleton 802 representing a patient, and inflatable sac locations on the front of the patient, R1 810A R2 810B R3 810C on a right side of the front of the patient, and L1 812A L2 812B L3 812C on a left side of the front of the patient.

In some embodiments, the pressure applicators (e.g., inflatable sacs) are optionally shaped as U-shaped sacs.

In some embodiments, the pressure applicators (e.g., inflatable sacs) are optionally placed, starting from the front of a patient, continuing laterally along a circumference of a torso of the patient, and continuing on a posterior side of the patient.

Reference is now made to FIGS. 9A-9C, which are simplified illustrations of one or more inflatable sacs in relation to a patient, in a system constructed and operational according to an example embodiment.

In FIGS. 9A-9C, an additional abdominal pressure applicator (e.g., inflatable sac) is provided. Such an abdominal pressure applicator potentially enables treating patients with neuromuscular disease and spinal cord injury who have impaired expiratory muscles.

FIGS. 9A-9C show an example abdominal inflatable sac, and a crescent or U shaped of some of the inflatable sacs.

FIGS. 9A-9C show non-limiting examples of a patient and inflatable sacs. FIGS. 9A-9C do not show the vest (such as shown in FIG. 1), so that the vest does not obscure a view of the sacs.

FIG. 9A shows a lateral view of a skeleton 902 representing a patient, and inflatable sacs R1 904A R2 904B R3 904C placed on example locations on a right side of the patient and an abdominal inflatable sac 905 placed against the patient's abdomen.

FIG. 9B shows a posterior view of the skeleton 902 representing a patient, and inflatable sac locations on the back of the patient, L1 906A L2 906B L3 906C on a left side of the back of the patient and R1 908A R2 908B R3 908C on a right side of the back of the patient.

FIG. 9C shows a frontal oblique view of the skeleton 902 representing a patient, and inflatable sac locations on the front of the patient, R1 910A R2 910B R3 910C on a right side of the front of the patient, L1 912A L2 912B L3 912C on a left side of the front of the patient, and an abdominal inflatable sac 911 placed against the patient's abdomen.

In some embodiments, the inflatable sacs are optionally shaped as U-shaped or crescent shaped sacs.

In some embodiments, an abdominal sac, for example as marked with “A” in FIGS. 9A and 9C, is optionally located on the upper abdomen, optionally between the rib cage and the umbilicus, from the anterior side. The abdominal sac potentially supports belly organs and potentially improves oxygenation by imitating the prone position.

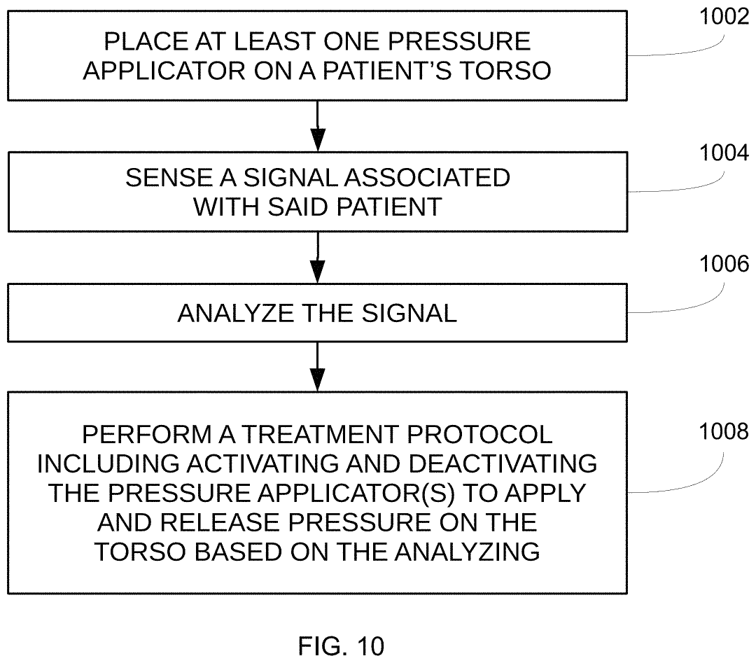

Reference is now made to FIG. 10, which is a simplified flow chart illustration of a method for providing treatment adapted to clear lung airways according to an example embodiment.

The method of FIG. 10 includes:
a. placing at least one pressure applicator on a patient's torso (1002);
b. sensing a signal associated with said patient (1004);
c. analyzing said signal (1006); and
d. performing a treatment protocol, said treatment protocol comprising activating and deactivating said pressure applicator to apply and release pressure on said torso, based, at least in part, on said analyzing said signal (1008).

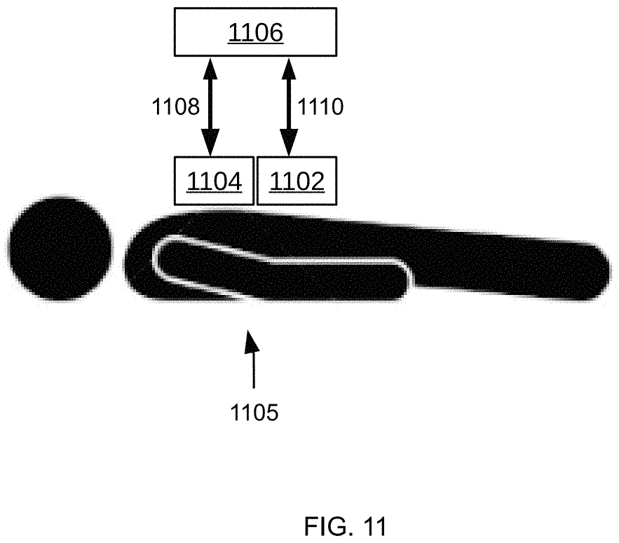

Reference is now made to FIG. 11, which is a simplified block diagram illustration of a system for providing treatment adapted to clear lung airways according to an example embodiment.

The system of FIG. 11 includes:

at least one pressure applicator 1102 adapted, when activated, to apply pressure at at least one specific location on a torso of a patient 1105; and, when deactivated, to release said pressure;

a sensor 1104 for sensing a signal associated with said patient 1105; and,

a controller 1106, in communication 1108 with said sensor 1104, adapted to analyze said signal and to control 1110 activation and deactivation of said pressure applicator 1102 based, at least in part, on analyzing said signal.

As used herein with reference to quantity or value, the terms “about” or “approximately” mean “within ±20% of”.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

1-50. (canceled)

51. A method for providing treatment adapted to clear lung airways, the method comprising;
placing at least one pressure applicator on a patient's torso; and
performing a treatment protocol, said treatment protocol comprising activating and deactivating said at least one pressure applicator to apply and release pressure on said torso,
wherein said treatment protocol comprises limiting the patient's breathing to different lung volumes at different breathing cycles,
thereby providing treatment to clear lung airways.

52. The method of claim 51 wherein said treatment protocol comprises:
applying pressure for a duration longer than a period of time corresponding to one of a group selected from:
said patient's inspiration; and
said patient's expiration.

53. The method of claim 51, wherein said treatment protocol comprises:
activating at least one pressure applicator located next to a top of the patient's torso, applying pressure thereon; and
activating at least one pressure applicator located next to a bottom of said patient's torso, applying pressure thereon.

54. The method of claim 51 and further comprising:
sensing a signal associated with said patient;
analyzing said signal; and
performing the treatment protocol based on the analyzing.

55. The method of claim 51, wherein said treatment protocol enables autogenic drainage.

56. The method of claim 51, comprising providing guidance to said patient to instruct said patient as to a desired breathing pattern.

57. The method of claim 56 wherein said guidance is synchronized to said activating and deactivating of said at least one pressure applicator.

58. The method of claim 54, comprising providing guidance to said patient to instruct said patient as to a desired breathing pattern, wherein said guidance is based on said sensing.

59. The method of claim 51, configured to induce coughing by deactivation of said at least one pressure applicator.

60. The method of claim 56 wherein said guidance comprises breathing instructions to said patient to a desired breathing pattern selected from a group consisting of:
long deep inspiration;
shallow inspiration;
prolonged expiration;
prolong inhalation;
quick breathing; and
a combination thereof.

61. The method of claim 54, wherein upon detection of a cough or intention to cough, at least one of said pressure applicators is deactivated.

62. The method of claim 51, comprising increasing a transpulmonary pressure gradient in said patient's lungs by rapid deactivation of at least one pressure applicator.

63. Airways clearance for a patient with a lung disease using the method of claim 51, wherein the lung disease is a lung disease selected from a group consisting of:
a chronic obstructive pulmonary disease (COPD);
a cystic fibrosis (CF) disease; and
a bronchiectasis disease.

64. A system for providing treatment adapted to clear lung airways, the system comprising:
at least one pressure applicator adapted, when activated, to apply pressure at at least one specific location on a torso of a patient; and, when deactivated, to release said pressure; and
a controller programmed to activate said at least one pressure applicator to limit the patient's breathing to different lung volumes at different breathing cycles,
thereby providing treatment to clear lung airways.

65. The system of claim 64 wherein said controller is programmed to activate said at least one pressure applicator for a duration longer than a period of time corresponding to one of a group selected from:
said patient's inspiration; and
said patient's expiration.

66. The system of claim 64 and further comprising:
a sensor for sensing a signal associated with said patient; and
said controller configured to:
receive and analyze said signal; and
perform the treatment protocol based on the analysis.

67. The system of claim 64, wherein said controller is adapted to provide guidance to a patient as to a required breathing pattern.

68. The system of claim 67 wherein said guidance is selected from a group consisting of:
written instructions;
voiced instructions;
sound instructions;
sensory instructions;
vibrations;
visual indications; and
a combination thereof.

69. The system of claim 64, wherein said controller receives data from a component selected from a group consisting of:
a BiPAP device;
an invasive ventilator;
non-invasive ventilator;
a cough simulating device; and
a combination thereof.

70. The system of claim 64, wherein release of pressure of said at least one pressure applicator is adapted to be provided in less than 0.5 seconds.
